(19) United States
(12) Patent Application Publication
(10) Pub. No.: US 2025/0235675 A1
(43) Pub. Date: Jul. 24, 2025

(54) SYSTEMS AND METHODS OF MONITORING A CATHETER OR SHUNT DEVICE AND FLUID OF THE SYSTEM

(71) Applicant: Bard Peripheral Vascular, Inc., Franklin Lakes, NJ (US)

(72) Inventors: Dawn M. HERRINGTON, Bixby, OK (US); Akiva W. KIRSCHNER, Chicago, IL (US); Christine T. KEARNEY, Palatine, IL (US); Nathan P. RETZLAFF, Kenosha, WI (US); Christopher J. SIMPSON, Gilbert, AZ (US); Wesley W. WALKER, Broken Arrow, OK (US)

(21) Appl. No.: 18/853,286

(22) PCT Filed: Apr. 7, 2023
(86) PCT No.: PCT/US2023/017860

§ 371 Date: Oct. 1, 2024

Related U.S. Application Data
(60) Provisional application No. 63/328,455, filed on Apr. 7, 2022.

(51) Int. Cl. A61M 27/00

(52) U.S. Cl. CPC A61M 27/002; A61M 2205/3303; A61M 2205/3523; A61M 2205/502; A61M 2210/101; A61M 2210/1017

(57) ABSTRACT

A monitoring system is provided. The monitoring system includes a controller and either a shunt device, an externally-communicating catheter, and/or a fluid retention device. The shunt device and catheter are configured to address fluid movement such as ascites and pleural effusions. Each of the shunt device and catheter include a plurality of sensors that communicate with the controller positioned external to a body of a subject. The plurality of sensors are configured to output a signal indicative of a total volume, a glucose amount, or an oxygenation within a fluid flowing in contact within or surrounding the shunt device or the catheter.

### CROSS REFERENCE TO RELATED APPLICATIONS

This utility patent application claims priority from U.S. Provisional patent application Ser. No. 63/328,455, filed on Apr. 7, 2022, and entitled “Systems and Methods of Wirelessly Monitoring A Shunt Device and Ascites Fluid of the System”, the entire contents of which is incorporated herein in its entirety.

### TECHNICAL FIELD

The present specification generally relates to systems and methods for monitoring the moving of fluid accumulations within a body such as pleural effusions and ascites and, more specifically, to a shunt and catheter devices and methods for wirelessly monitoring fluid movement and the function thereof.

### BACKGROUND

Devices, such as shunt device and catheters, are used to drain or move fluid from an area within the body to another area. Specifically, a peritoneovenous shunt device is a fully implanted shunt that is used to move excess fluid from the peritoneal cavity to the venous system thereby palliating the symptoms associated with recurrent ascites. A pleuro-peritoneal shunt device is a fully implanted shunt device that transfers fluid from the pleural cavity to the peritoneum thereby palliating the symptoms associated with recurrent pleural effusion. Further, a peritoneal catheter is a partially implanted catheter used to move excess fluid from the peritoneal cavity to outside the body for disposal thereby palliating the symptoms associated with recurrent ascites. A pleural catheter is a partially implanted device that transfers fluid from the pleural cavity to outside the body for disposal thereby palliating the symptoms associated with recurrent pleural effusion. Ascites is the accumulation of fluid within the peritoneal cavity and pleural effusion is the accumulation of fluid within the pleural space. The currently known peritoneovenous shunt device include a fenestrated peritoneal catheter, a flexible pump chamber containing either one or two miter valves, and a venous catheter. The currently known pleuro-peritoneal shunt device include a fenestrated pleural catheter, a flexible pump chamber containing either one or two miter valves, and a fenestrated peritoneal catheter. The peritoneal and pleural catheters include a fenestrated portion of the catheter that is fully implanted in the bodily cavity containing fluid, a subcutaneously tunneled portion of the catheter with a cuff for tissue ingrowth, and an external portion of the catheter that is intermittently accessed by the patient or caregiver to remove the fluid. The shunt devices move the ascites fluid from the peritoneal or pleural cavity through the one-way miter valves, which are designed to open when the system experiences a pressure differential of 3 cm of water or may be actively pumped due to the negative pressure of the pleural space. However, none of the known devices provide data to indicate to a physician how well the shunt devices or catheter function or data of the fluid in contact with the shunt devices or catheter.

Accordingly, there is a need to provide assistance to a physician in determining how well the shunt device or catheter is performing and/or the type of fluid in contact with the shunt device or catheter.

### SUMMARY

In one aspect, a wireless monitoring system is provided. The wireless monitoring system includes a controller and a shunt device. The controller is configured to be positioned external to a subject. The shunt device is configured to be implanted within the user. The shunt device has a pump chamber and a pair of limbs. The pump chamber or the pair of limbs further include one or more wireless sensors communicatively coupled to the controller. The at least one or more wireless sensors configured to output a signal indicative of a total volume, a glucose amount, or an oxygenation within a fluid flowing in contact with the pump chamber or at least one of the pair of limbs. The one or more wireless sensors wirelessly transmit a data to the controller.

In another aspect, a pleuro-peritoneal shunt assembly for fluidly coupling a pleural cavity to a peritoneal cavity of a subject is provided. The pleuro-peritoneal shunt assembly includes a controller and a shunt device. The controller is configured to be positioned external to the subject. The shunt device is configured to be embedded within the subject and in wireless communication with the controller. The shunt device includes a pair of limbs and a pump chamber. One of the pair of limbs fluidly coupled to one side of the pump chamber and the other one of the pair of limbs fluidly coupled to an opposite side of the pump chamber. The pump chamber or the pair of limbs include one or more wireless sensors communicatively coupled to the controller. At least one of the one or more wireless sensors configured to output a signal indicative of a total volume, a glucose amount, or an oxygenation of a fluid in contact with the pump chamber or in contact with one of the pair of limbs. The one or more wireless sensors wirelessly transmit a plurality of data to the controller.

In yet another aspect, a peritoneovenous shunt assembly for fluidly coupling a peritoneal cavity to a venous system of a subject is provided. The peritoneovenous shunt assembly includes a controller and a shunt device. The controller is configured to be positioned external to the subject. The shunt device is configured to be embedded within the subject and in wireless communication with the controller. The shunt device includes a pair of limbs and a pump chamber. One of the pair of limbs fluidly coupled to one side of the pump chamber and the other one of the pair of limbs fluidly coupled to an opposite side of the pump chamber. The pump chamber or the pair of limbs include one or more wireless sensors communicatively coupled to the controller. At least one of the one or more wireless sensors configured to output a signal indicative of a total volume, a glucose amount, or an oxygenation of a fluid within the pump chamber or within one of the pair of limbs. The one or more wireless sensors wirelessly transmit a plurality of data to the controller.

In yet another aspect, a monitoring system is provided. The monitoring system includes a controller and a catheter. The controller is configured to be positioned external to a subject. The catheter is configured to be implanted within the subject. The catheter has a distal end and an opposite proximal end, and an external valve portion positioned at the proximal end. The external valve portion includes one or more sensors communicatively coupled to the controller. At least one or more sensors is configured to output a signal indicative of a total volume, a glucose amount, or an oxygenation within a fluid flowing in contact with the external valve portion or within the catheter. The one or more sensors transmit data to the controller.

In yet another aspect, a pleural catheter assembly for fluidly coupling a pleural cavity to an external fluid retention device is provided. The pleural catheter assembly includes a controller and a catheter. The controller is configured to be positioned external to a subject. The pleural catheter includes a distal end, an opposite proximal end, and an external valve portion fluidly coupled to the proximal end. The external valve portion includes one or more sensors communicatively coupled to the controller. At least one of the one or more sensors is configured to output a signal indicative of a total volume, a glucose amount, or an oxygenation of a fluid passing through the catheter. The distal end is configured to be embedded within the subject. The external valve portion is configured to be in communication with the controller such that the one or more sensors transmit a plurality of data to the controller.

In yet another aspect, a peritoneal catheter assembly for fluidly coupling a peritoneal cavity to an external fluid retention device is provided. The peritoneal catheter assembly includes a controller, a peritoneal catheter, and an external valve portion. The controller is configured to be positioned external to a subject. The peritoneal catheter includes a distal end, an opposite proximal end. The external valve portion is fluidly coupled to the proximal end. The external valve portion includes one or more sensors communicatively coupled to the controller. At least one of the one or more sensors is configured to output a signal indicative of a total volume, a glucose amount, or an oxygenation of a fluid passing through the catheter. The distal end is configured to be embedded within the subject. The external valve portion is configured to be in communication with the controller such that the one or more sensors transmit a plurality of data to the controller.

In yet another aspect, a monitoring system is provided. The monitoring system includes a controller, a catheter, an external fluid retention device, and a dock device. The controller is configured to be positioned external to a subject. The catheter has a distal end and an opposite proximal end. The distal end is configured to be implanted within the subject. The external fluid retention device is fluidly coupled to the proximal end of the catheter. The external fluid retention device has a negative pressure. The dock device has a pressure sensor configured to sense a pressure within the catheter or within the external fluid retention device. The dock device in wireless communication with the controller to transmit data to the controller.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of an example shunt device system, according to one or more embodiments shown or described herein;

FIG. 2A schematically depicts the example shunt device system of FIG. 1 arranged as a peritoneovenous shunt device within a subject, according to one or more embodiments shown or described herein;

FIG. 2B schematically depicts the example shunt device system of FIG. 1 arranged as a pleuro-peritoneal shunt device within the subject, according to one or more embodiments shown or described herein;

FIG. 3A schematically depicts an isolated view of a pump chamber and portion of a pair of limbs of the shunt device of FIG. 1 with a plurality of sensors positioned within the pump chamber, according to one or more embodiments shown or described herein;

FIG. 3B schematically depicts another isolated view of a pump chamber and portion of a pair of limbs of the shunt device of FIG. 1 with the plurality of sensors positioned within the pair of limbs, according to one or more embodiments shown or described herein;

FIG. 4 depicts a block diagram of illustrative components of the shunt device system of FIG. 1, according to one or more embodiments shown or described herein

FIG. 5 schematically depicts an example circuit of the shunt device system of FIG. 1, according to one or more embodiments shown or described herein;

FIG. 6 depicts a flowchart of an illustrative method of using the shunt device system of FIG. 1, according to one or more embodiments shown or described herein;

FIG. 7A schematically depicts a perspective view of a first aspect of a catheter device system, according to one or more embodiments shown or described herein;

FIG. 7B, schematically depicts a partial cross section of the catheter of FIG. 7A taken from line 7B-7B, according to one or more embodiments shown or described herein;

FIG. 7C schematically depicts a perspective view of a second aspect of the catheter device system of FIG. 7A, according to one or more embodiments shown or described herein;

FIG. 8A schematically depicts the catheter device system of FIG. 7A arranged as a pleural catheter device positioned within a subject, according to one or more embodiments shown or described herein;

FIG. 8B schematically depicts the catheter device system of FIG. 7A arranged as a peritoneal catheter device positioned within the subject, according to one or more embodiments shown or described herein;

FIG. 9 depicts a flowchart of an illustrative method of using the catheter device system of FIG. 7A, according to one or more embodiments shown or described herein;

FIG. 10A schematically depicts a front view of a wireless drained volume and catheter device monitoring system that includes a dock positioned in line with a fluid flow path, according to one or more embodiments shown or described herein;

FIG. 10B schematically depicts a front view of the wireless drained volume and catheter device monitoring system of FIG. 10A with the dock positioned out of line of the fluid flow path, according to one or more embodiments shown or described herein;

FIG. 10C schematically depicts a front view of the wireless drained volume and catheter device monitoring system of FIG. 10A with the dock positioned as a top for an external fluid retention device, according to one or more embodiments shown or described herein;

FIG. 10D schematically depicts a front view of the wireless drained volume and catheter device monitoring system of FIG. 10A with the dock integrated into a flow control, according to one or more embodiments shown or described herein; and

FIG. 10E schematically depicts a front view the wireless drained volume and catheter device monitoring system of FIG. 10A with the dock integrated into a catheter hub, according to one or more embodiments shown or described herein.

### DETAILED DESCRIPTION

Embodiments herein are directed to wireless shunt device monitoring systems, catheter device monitoring systems, and wireless drained volume and catheter device monitoring systems. In embodiments, the wireless shunt device monitoring system includes a fully implantable shunt device, such as a peritoneovenous shunt device or a pleuro-peritoneal shunt device, an electronic control unit, and a display device. The shunt device generally includes flexible pump chamber that includes at least one miter valve, a first limb, such as a fenestrated peritoneal catheter, extending from one end of the pump chamber, and a second limb, such as a venous catheter, extending from the opposite end of the pump chamber. The shunt device further includes a plurality of sensors positioned within the pump chamber and/or within the first and second limbs. The plurality of sensors gather data and transmit data (e.g., wirelessly) to be monitored by the electronic control unit either continuously or on-demand.

In embodiments, the catheter device monitoring system includes a catheter with a distal end that is fully implantable, such as a peritoneal catheter or a pleural catheter, the electronic control unit, and the display device. The catheter further includes a proximal end that may further include an external valve portion. The catheter further includes a plurality of sensors positioned within the external valve portion and/or within the catheter positioned between the distal end and the proximal end. The plurality of sensors gather data and transmit data (e.g., either through wired or wireless communication channels) to be monitored by the electronic control unit either continuously or on-demand.

The electronic control unit may receive data from the plurality of sensors to monitor various aspects of the subject's physiology and fluid accumulations that may be useful in monitoring the function of the shunt device, the catheter, and/or underlying health of the subject. Example data may include flow rate data, total volume throughput, differential pressure, temperature, glucose, pH/oxygenation, proteins, albumin, and/or the like. The data may be tracked external to the subject via a display device, that is, the data may be displayed, such as in real-time, on a display. Moreover, in some embodiments, the plurality of sensors of the shunt device, catheter, and/or external fluid retention device may be powered via inductive coupling to the controller, or other device. As such, the plurality of sensors may be independent from any internal requirement of a power source, wired communication with the controller, or the like.

In embodiments, the wireless drained volume and catheter device monitoring system includes a controller, a catheter, an external fluid retention device, and a dock device. The external fluid retention device is fluidly coupled to the proximal end of the catheter and is configured to receive fluid from the subject via a negative pressure of the external fluid retention device. The dock device has a pressure sensor configured to sense a pressure within the catheter or within the external fluid retention device. The dock device may be in wireless communication with the controller to transmit data to the controller.

Each of the above embodiments, provide improvement over prior systems by allowing users to monitor device performance. Various embodiments and benefits of the monitoring systems will be described in greater detail herein.

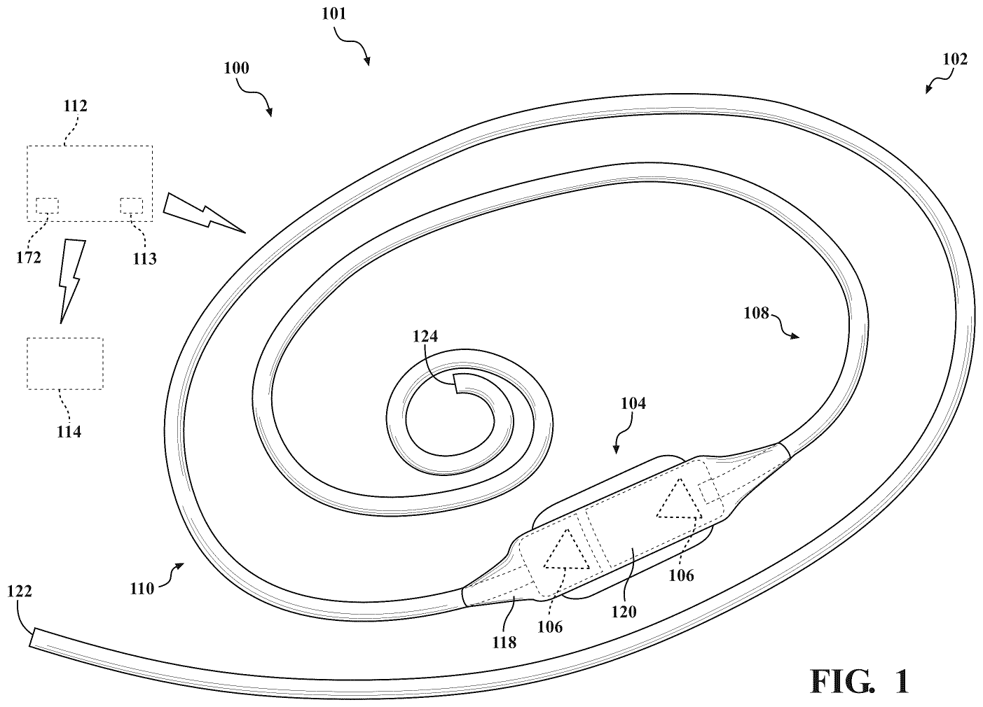

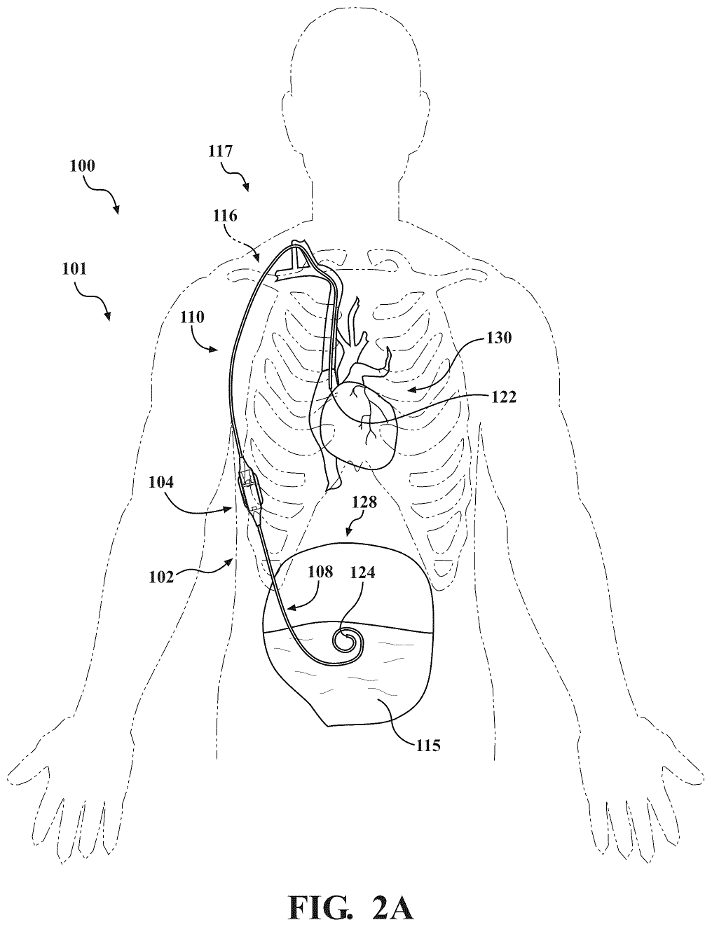

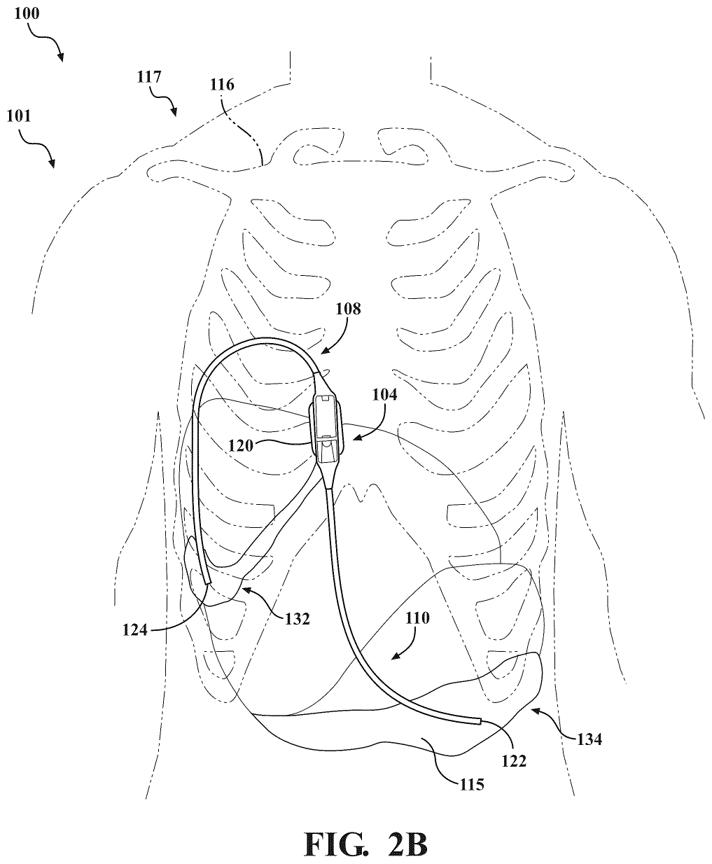

Referring now to FIGS. 1 and 2A-2B, a device monitoring system 100 is generally depicted. In the present embodiment, the example device monitoring system 100 is a wireless shunt device monitoring system 101. As illustrated, the wireless shunt device monitoring system 101 may include a shunt device 102 that includes a pump chamber 104, at least one valve 106, a first limb 108 or catheter, and a second limb 110 or catheter. The wireless shunt device monitoring system 101 may further include a controller 112, which may include an electronic control unit 113, a display device 114, and a power device 172, though a greater or fewer number of components are contemplated and possible. As will be described in greater detail herein, the shunt device 102 may be configured, as illustrated in FIG. 2A, as a peritoneovenous shunt device to passively move fluids 115 from a peritoneal cavity 128 to a venous system 130 thereby palliating the symptoms associated with recurrent ascites. The shunt device 102 may also be configured, as illustrated in FIG. 2B, as a pleuro-peritoneal shunt device to transfer fluids 115 from a pleural cavity 132 to a peritoneum 134 thereby palliating the symptoms associated with recurrent pleural effusion.

It is noted that the shunt device 102 may be used to move or transport any type of fluid from and to any type of cavity/area within a body 116 of a subject 117. For example, the shunt device 102 may be used to move or transport ascites fluids, effusion fluids, and the like. Further, the shunt device 102 is not limited to the peritoneovenous shunt device or the pleuro-peritoneal shunt device, but instead may be configured to transport any type of fluid from and to any type of cavity/area within a body 116 of a subject 117 such as from to the bladder to other areas of the body, such as the central venous system. As another non-limiting example, fluids that the shunt device 102 move or transport may include pericardial effusions pus, cerebro-spinal fluid, bile, urine, and the like.

Referring still to FIG. 1, in some embodiments, the pump chamber 104 may be configured to pump fluid through the shunt device 102. For example, the pump chamber 104 may include a housing 118, a flexible membrane 120, and the at least one valve 106. It is noted that the pump chamber 104 may include a greater or fewer number of components without departing from the scope of the present disclosure.

The housing 118 generally provides the structure of the pump chamber 104 to which the various components are connected. The housing 118 may be flexible or rigid. In embodiments, the housing 118 defines own or more fluid pathways or chambers through which the ascites fluid may be directed.

In some embodiments, the housing 118 may seal a motor, control unit, and battery from an outside environment, such as inside of the subject's body 116. In this embodiment, the housing 118 may be made from a non-conductive material to allow for wireless communication with the control unit and induction charging of the battery.

The flexible membrane 120 may be coupled to the housing 118 and may be used to manually pump fluids, such as the fluids 115 through the shunt device 102 by depressing the flexible membrane 120. For example, the flexible membrane 120 may act as a button and by depressing the flexible membrane 120, a pressure may be created within the housing 118, which causes the fluids 115 contained within the shunt device 102 to move into and from the first limb 108 into and through the second limb 110. That is, the fluids 115 flow from into and through the first limb 108, into the housing 118 of the pump chamber 104, through the at least one valve 106, and into and through the second limb 110.

Accordingly, the at least one valve 106 may be positioned within the housing 118 and operable to prevent back-flow of any fluids 115 passing through the pump chamber 104 from flowing back toward an originating site of the fluids 115. The at least one valve 106 may be a mitre valve, a duckbill valve, or another type of one-way valve which only allows fluids 115 to flow in a single direction when a pressure differential is created on opposite sides of the at least one valve 106. The at least one valve 106 may include any number of valves such as one or more valves, two or more valves, three or valves, and/or the like.

Now referring collectively to FIGS. 1 and 2A-2B, the first limb 108 may be coupled to the housing 118 so as to be in fluid communication with the at least one valve 106. The first limb 108 may generally include a tip portion 124, which, for example, may be inserted into the peritoneal cavity 128 of the body 116 of the subject 117, as best illustrated in FIG. 2A or the pleural cavity 132 of the body 116 of the subject 117, as best illustrated in FIG. 2B. The first limb 108 may be any type of flexible tubing suitable to be inserted within the subject's body 116. Additionally, in embodiments, the first limb 108 may be shaped in various forms in order to secure the first limb 108 within the peritoneal cavity 128 or the pleural cavity 132. For example, the tip portion 124 may be bent in a curve or spiral shaped in order to secure or anchor the tip portion 124 within the peritoneal cavity 128 or the pleural cavity 132.

Arranged on the opposite side of the at least one valve 106 and coupled to the housing 118 so as to be in fluid communication with the at least one valve 106 (e.g., downstream of the at least one valve 106) may be the second limb 110. The second limb 110 may generally include a tip portion 122 which may be, for example, inserted into the venous system 130 of the subject 117, as best illustrated in FIG. 2A, or to the peritoneum (peritoneal cavity) 134 of the subject 117, as best illustrated in FIG. 2B. The second limb 110 may be any type of flexible tubing suitable to be inserted within the subject's body 116. Additionally, in embodiments, the second limb 110 may be shaped in various forms in order to secure the second limb 110 within the venous system 130 or the peritoneum (peritoneal cavity) 134. For example, as discussed above, the tip portion 122 may be bent in a curve or spiral shaped in order to secure or anchor the tip portion 122 within the venous system 130 or the peritoneum (peritoneal cavity) 134.

Referring specifically to FIG. 2A, the shunt device 102 may be a peritoneovenous shunt device that passively moves fluids 115 from the peritoneal cavity 128 to the venous system 130 thereby palliating the symptoms associated with recurrent ascites. For example, without limitation, ascites fluids. The shunt device 102 may be a fully implanted shunt arranged within the body 116 of a subject 117 in order to move or transport the fluids 115 from the peritoneal cavity 128 to the venous system 130. Specifically, the tip portion 124 and portions of the first limb 108 may be arranged within the peritoneal cavity 128 of the body 116 of the subject 117. The peritoneal cavity 128 is a space between the parietal peritoneum (the peritoneum that surrounds the abdominal wall) and the visceral peritoneum (the peritoneum that surrounds the internal organs) within the body 116 of the subject 117. However, in embodiments, the first limb 108 may be arranged in any area of the body 116, which requires a fluid, such as ascites fluids, to be moved or transported. The first limb 108 may be arranged within the peritoneal cavity 128 in such a way that the fluids 115 may enter the first limb 108. For example, the tip portion 124 of the first limb 108 may be arranged in the lower section of the peritoneal cavity 128 where the fluids 115 collects within the peritoneal cavity 128. The first limb 108 may extend through an incision within the wall of the peritoneal cavity 128. The pump chamber 104 may be arranged outside of the peritoneal cavity 128, and more specifically, may be arranged just underneath the skin of the body 116 to allow for the subject 117 to depress the flexible membrane 120 of the pump chamber 104 and for wireless charging or communication with the controller 112, as discussed in greater detail herein.

Still referring still to FIG. 2A, the second limb 110 may be arranged within the venous system 130 of the subject 117. As the fluids 115 may be transported from the peritoneal cavity 128 to the venous system 130 through the shunt device 102, the venous system 130 accepts and diffuses the fluids 115 through various techniques, such as osmoses, diffusion, or the like. As noted above, the at least one valve 106 may prevent backflow of the fluid from the venous system 130 to the pump chamber 104.

Due to the arrangement of the shunt device 102 within the body 116 of the subject 117, with the first limb 108 arranged within the peritoneal cavity 128 and the second limb 110 arranged within the venous system 130, a passive pressure-driven flow of fluids 115 may be formed between the peritoneal cavity 128 and the venous system 130 through the shunt device 102. Since the peritoneal cavity 128 and the venous system 130 are fluidly coupled, any pressure gradient between the peritoneal cavity 128 and the venous system 130 will create a pressure-driven flow of fluid. For example, as pressure increases within the peritoneal cavity 128, the fluid stored within the peritoneal cavity 128 may be forced through the first limb 108 as a pressure release for the peritoneal cavity 128. The pressure within the peritoneal cavity 128 may be increased by having the subject exert their diaphragm into their abdominal cavity, or by breathing normally. Since the action of breathing or exerting a diaphragm does not increase the pressure within the venous system 130, a pressure gradient may be formed between the peritoneal cavity 128 and the venous system 130, with the high pressure being present in the peritoneal cavity 128, and the low pressure being present in the venous system 130. This difference in pressures passively flows the fluids 115 from the peritoneal cavity 128 to the venous system 130, without the need of additional pumping mechanisms. Due to the arrangement of the pump chamber 104 being in fluid communication, the passive pressure driven fluids, such as the fluids 115 may still flow through the shunt device 102 even when the pump is not activated. That is, when the subject 117 manually depresses on the flexible membrane 120 of the housing 118, a differential in the housing 118 may be formed causing the fluids 115 to move from the peritoneal cavity 128 to the venous system 130. Additionally, even when the pump chamber 104 is not active, the system may passively move or transport the fluids 115 from the peritoneal cavity 128 to the venous system 130.

Referring now to FIG. 2B, the shunt device 102 may be a pleuro-peritoneal shunt device that passively moves fluids 115 from the pleural cavity 132 to the peritoneum (peritoneal cavity) 134 thereby palliating the symptoms associated with recurrent pleural effusion. For example, without limitation, effusion fluids. The shunt device 102 may be a fully implanted shunt arranged within the body 116 of a subject 117 in order to move or transport the fluids 115 from the pleural cavity 132 to the peritoneum (peritoneal cavity) 134. Specifically, the tip portion 124 and portions of the first limb 108 may be arranged within the pleural cavity 132 of the body 116 of the subject 117. The pleural cavity 132 is a space that is enclosed by the spine, ribs, and sternum (breast bone) and is separated from the abdomen by the diaphragm and the peritoneum 134 is the tissue that lines your abdominal wall and covers most of the organs in your abdomen within the body 116 of the subject 117. However, in embodiments, the first limb 108 may be arranged in any area of the body 116, which requires a fluid to be moved or transported. The first limb 108 may be arranged within the pleural cavity 132 in such a way that the fluids 115 may enter the first limb 108. For example, the tip portion 124 of the first limb 108 may be arranged in the lower section of the pleural cavity 132 where the fluids 115 collect within the pleural cavity 132. The first limb 108 may extend through an incision within the wall of the pleural cavity 132. The pump chamber 104 may be arranged outside of the pleural cavity 132, and more specifically, may be arranged just underneath the skin of the body 116 to allow for the subject 117 to depress the flexible membrane 120 of the pump chamber 104 and for wireless charging or communication with the controller 112, as discussed in greater detail herein.

Still referring still to FIG. 2B, the second limb 110 may be arranged within the peritoneum (peritoneal cavity) 134 of the subject 117. As the fluids 115 may be transported from the pleural cavity 132 to the peritoneum 134 through the shunt device 102, the peritoneum (peritoneal cavity) 134 accepts and diffuses the fluids 115 through various techniques, such as osmoses, diffusion, or the like. The at least one valve 106 prevents backflow of the fluids 115 from the peritoneum (peritoneal cavity) 134 to the pump chamber 104.

Due to the arrangement of the shunt device 102 within the body 116 of the subject 117, with the first limb 108 arranged within the pleural cavity 132 and the second limb 110 arranged within the peritoneum (peritoneal cavity) 134, a passive pressure-driven flow of fluids 115 may be formed between the peritoneum (peritoneal cavity) 134 and the pleural cavity 132 through the shunt device 102. Since the peritoneum (peritoneal cavity) 134 and the pleural cavity 132 are fluidly coupled, any pressure gradient between the peritoneum (peritoneal cavity) 134 and the pleural cavity 132 will create a pressure-driven flow of fluids 115. For example, as pressure increases within the pleural cavity 132, the fluids 115 stored within the pleural cavity 132 will be forced through the first limb 108 as a pressure release for the pleural cavity 132. The pressure within the pleural cavity 132 may be increased by having the subject 117 exert their diaphragm into their abdominal cavity, or by breathing normally. Since the action of breathing or exerting a diaphragm does not increase the pressure within the pleural cavity 132, a pressure gradient may be formed between the pleural cavity 132 and the pleural cavity 132, with the high pressure being present in the pleural cavity 132, and the low pressure being present in the pleural cavity 132. This difference in pressures passively flows the fluids 115 from the pleural cavity 132 to the peritoneum (peritoneal cavity) 134, without the need of additional pumping mechanisms. Due to the arrangement of the pump chamber 104 being in fluid communication, the passive pressure driven fluids flow, such as the fluids 115 may still flow through the shunt device 102 even when the pump is not activated. That is, when the subject 117 manually depresses on the flexible membrane 120 of the housing 118, a differential in the pump may be formed causing the fluids 115 to move from the pleural cavity 132 to the peritoneum (peritoneal cavity) 134. Additionally, even when the pump chamber 104 is not active, the system may passively move or transport the fluids 115 from the pleural cavity 132 to the peritoneum (peritoneal cavity) 134.

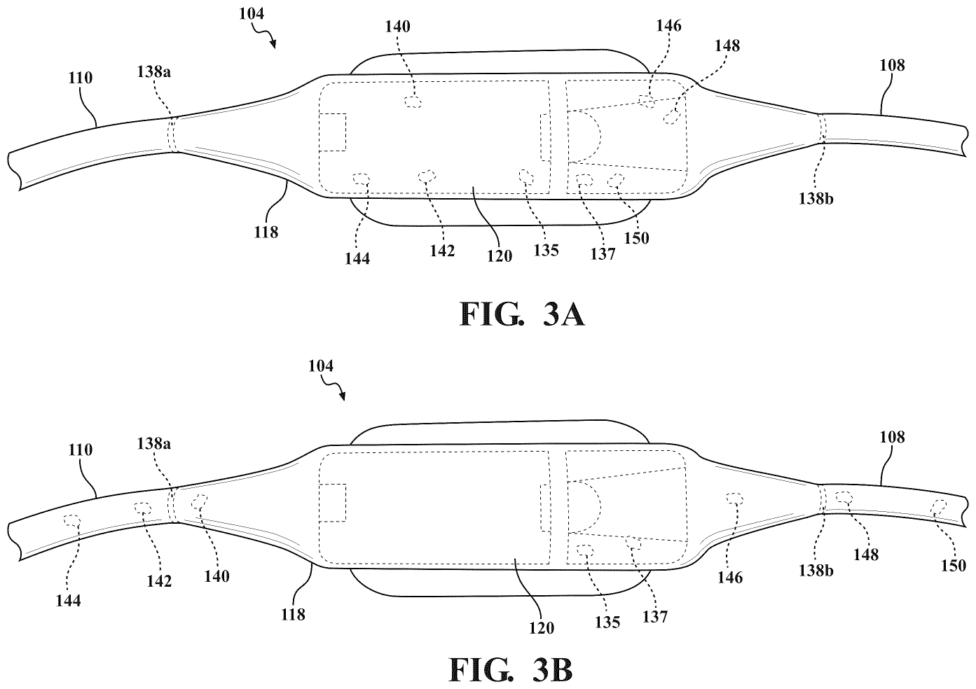

Now referring to FIG. 3A, an isolated view of the pump chamber 104 and a portion of each of the first and second limbs 108, 110 is schematically depicted. In embodiments, the shunt device 102 further includes one or more sensors such as a plurality of sensors 152. The plurality of sensors 152 may include a number of various sensors that will be described in greater detail herein.

For example, the first and second limbs 108, 110 may each include flow meters 138a, 138b. Each of the flow meters 138a, 138b may be strategically positioned on each side of the pump chamber 104 (e.g., upstream and downstream of the pump chamber 104 with respect to the flow of fluids 115 (FIGS. 2A-2B). Each of the flow meters 138a, 138b may be communicatively coupled to the controller 112 (depicted in FIG. 1) such as via any wireless communication protocol (e.g., Bluetooth, cellular, or similar technology). Data gathered by the flow meters 138a, 138b may be gathered and transmitted to the controller 112. As such, the controller 112 may analyze the data wirelessly received from the each of the flow meters 138a, 138b to determine or calculate a differential flow rate between the pair of limbs 110 or within the pump chamber 104. A variance in the differential flow rate may indicate potential clogging within the shunt device 102, such as at the tip portion 124 of the first limb 108, and/or at the tip portion 122 of the second limb 110. It should be appreciated that the flow meters 138a, 138b need not be positioned within the first limb 108 and/or the second limb 110 and instead may be positioned to gather data from the fluid in contact with the first limb 108 and/or the second limb 110 regardless of the fluid flow through the pump chamber 104, the first limb 108 and/or the second limb 110 or a fluid flow that surrounds the first limb 108 and/or the second limb 110 external to the pump chamber 104, the first limb 108 and/or the second limb 110.

Example flow meters 138a, 138b may include implantable flow sensors. Implantable flow sensors may be used to monitor the flow rate of fluids 115 (FIGS. 2A-2B) through tubular walls such within the first and second limbs 108, 110. In some embodiments, implantable flow sensors may be fitted around the tubular wall of the first and second limbs 108, 110 external to the flow path. Further, implantable flow sensors may use, for example and not limited to, electromagnetic, ultrasonic, and time of flight techniques where the electromagnetic sensors generate a magnetic field perpendicular to the flow of the fluids 115 (FIGS. 2A-2B) through the first and second limbs 108, 110 such that charged ions in the fluids 115 (FIGS. 2A-2B) pass through the magnetic field causing variation in the conductivity as measured by an electrode. Other example flow meters 138a, 138b may include, but are not limited to, ultrasonic flow sensors, micromachined flow sensors, membrane-less mass flow micro sensors, carbon nanotubes for sensing applications, thermal flow sensors, MEMS flow sensors, digital sensors for fluids flow, vortex-shedding sensors, and/or the like.

As discussed in greater detail herein, the pump chamber 104 may also include or house one or more of the plurality of sensors 152. For example, the pump chamber 104 may include a pressure sensor 140, a temperature sensor 142, a glucose sensor 144, a pH sensor 146, a total volume sensor 148, an oxygen concentration sensor 150, or the like. The flow meters 138a, 138b, the pressure sensor 140, the temperature sensor 142, the glucose sensor 144, the pH sensor 146, the total volume sensor 148, and/or the oxygen concentration sensor 150, collectively, may be referred to as the plurality of sensors 152. In embodiments, each of the pressure sensor 140, the temperature sensor 142, the glucose sensor 144, the pH sensor 146, the total volume sensor 148, and/or the oxygen concentration sensor 150 may be positioned within the housing 118 such as below the flexible membrane 120 of the pump chamber 104. The wireless communication protocols between the control unit 112 and the plurality of sensors 152 will be discussed in greater detail below.

The pressure sensor 140 may include any sensor configured to output a signal indicative of a pressure within the pump chamber 104. For example, the pressure sensor 140 may use a left ventricular assist device (LVAD) technology to detect and transmit data (e.g., wirelessly) related to the perfusion of the fluids 115 (FIGS. 2A-2B) within the pump chamber 104. In other embodiments, the pressure sensor 140 may be a potentiometric type pressure sensor, inductive type pressure sensors, capacitive type pressure sensors, piezoelectric type pressure sensors, strain gauge type pressure sensors, variable reluctance type pressure sensors, and/or the like.

It should be appreciated that the pressure sensor 140 is not limited to be positioned within the pump chamber 104 or output a signal indicative of a pressure within the pump chamber 104. The pressure sensor 140 may be positioned anywhere to gather data from the fluid in contact with the pump chamber 104 regardless of the fluid flowing through the pump chamber 104 or fluid flow that surrounds the pump chamber 104 external to the pump chamber 104. For example, the pressure sensor 140 may be positioned in the first limb 108 and/or on the first limb 108, in the second limb 110 and/or on the second limb 110, in the housing 118 and/or on the housing 118, or the like.

The temperature sensor 142 may include any sensor configured to output a signal indicative of a temperature of a fluid within the pump chamber 104 or within the first limb 108 and/or the second limb 110. For example, the temperature sensor 142 may be a wireless fibre optic temperature probe that detects and transmits data related to the temperature of the fluids 115 (FIGS. 2A-2B) moving through the pump chamber 104, the first limb 108 and/or through the second limb 110. In other embodiments, the temperature sensor 142 may a thermocouple type sensor, a resistance type temperature detector, semiconductor type temperature sensors, and/or the like.

It should be appreciated that the temperature sensor 142 need not be positioned within the pump chamber 104, the first limb 108 and/or the second limb 110 and instead may be positioned external to the shunt device 102 to gather data from the fluid in contact with the pump chamber 104, the first limb 108 and/or the second limb 110 regardless of the fluid flowing through the pump chamber 104, the first limb 108 and/or the second limb 110 and/or a fluid flow that surrounds the pump chamber 104, the first limb 108 and/or the second limb 110.

The glucose sensor 144 may include any sensor configured to output a signal indicative of a glucose level within a fluid within the pump chamber 104, the first limb 108 and/or the second limb 110. For example, the glucose sensor 144 may be an implantable type sensor, such as those that include a substrate with a sensor, an RF detection circuit, a glucose affinity polymer and an antenna. As such, the glucose sensor detects and transmits data (e.g., wirelessly) related to glucose in the fluids 115 (FIGS. 2A-2B) moving through the pump chamber 104, the first limb 108 and/or the second limb 110. The glucose sensor may use different techniques of glucose recovery such as reverse iontophoresis, direct subcutaneous implantation and microdialysis.

It should be appreciated that the glucose sensor 144 need not be positioned within the pump chamber 104, the first limb 108 and/or the second limb 110 and instead may be positioned to gather data from the fluid in contact with the pump chamber 104, the first limb 108 and/or the second limb 110 regardless of the fluid flowing through the pump chamber 104, the first limb 108 and/or the second limb 110 and/or the fluid flow that surrounds the pump chamber 104, the first limb 108 and/or the second limb 110 external to the pump chamber 104, the first limb 108 and/or the second limb 110. For example, the glucose sensor 144 may be positioned in the first limb 108 and/or on the first limb 108, in the second limb 110 and/or on the second limb 110, in the housing 118 and/or on the housing 118, or the like.

The pH sensor 146 and the oxygen concentration sensor 150 may include any sensor or multiple sensors configured to output a signal indicative of an oxygen concentration and/or pH level within a fluid within the pump chamber 104 the first limb 108 and/or the second limb 110. For example, the pH sensor 146 and the oxygen concentration sensor 150 may be included in a single sensor assembly or may be independent sensors. The pH sensor 146 and/or the oxygen concentration sensor 150 detect and transmit data (e.g., wirelessly) related to the pH and oxygen concentration, respectively, of the fluids 115 (FIGS. 2A-2B) moving through the pump chamber 104, the first limb 108 and/or the second limb 110. In some embodiments, combination sensors may use amperometric, potentiometric and/or impedance sensors for measurement of the oxygen concentration, the pH value and the impedance in the fluids 115 (FIGS. 2A-2B) moving through the pump chamber. The various electronic circuits are designed for maximum energy efficiency to allow, together with the high integration density, as small as possible and wireless active implants.

It should be appreciated that the pH sensor 146 and the oxygen concentration sensor 150 need not be positioned within the pump chamber 104, the first limb 108 and/or the second limb 110 and instead may be positioned to gather data from the fluid in contact with the pump chamber 104, the first limb 108 and/or the second limb 110 regardless of the fluid flowing through the pump chamber 104, the first limb 108 and/or the second limb 110 and/or the fluid flow that surrounds the pump chamber 104, the first limb 108 and/or the second limb 110 external to the pump chamber 104, the first limb 108 and/or the second limb 110. For example, the pH sensor 146 may be positioned in the first limb 108 and/or on the first limb 108, in the second limb 110 and/or on the second limb 110, in the housing 118 and/or on the housing 118, or the like.

The total volume sensor 148 may include any sensor configured to output a signal indicative of a total volume of fluid passing through the pump chamber 104, the first limb 108 and/or the second limb 110, and transmit data (e.g., wirelessly) related to total volume of fluids 115 (FIGS. 2A-2B) moving through the pump chamber 104, the first limb 108 and/or the second limb 110. As such, the total volume sensor 148 may assist the controller 112 in detecting undesirable conditions such as increased fluids in various cavities. In some embodiments, the total volume sensor 148 may be ultrasonic type flow sensors, micromachined type flow sensors, membrane-less mass flow micro type sensors, carbon nanotubes type for sensing applications, thermal type flow sensors, MEMS type flow sensors, digital type sensors for fluids 115 (FIGS. 2A-2B) flow, vortex-shedding type sensors, and/or the like.

It should be appreciated that the total volume sensor 148 need not be positioned within the pump chamber 104, the first limb 108 and/or the second limb 110 and instead may be positioned to gather data from the fluid in contact with the pump chamber 104, the first limb 108 and/or the second limb 110 regardless of the fluid flowing through the pump chamber 104, the first limb 108 and/or the second limb 110 and/or the fluid flow that surrounds the pump chamber 104, the first limb 108 and/or the second limb 110 external to the pump chamber 104, the first limb 108 and/or the second limb 110. For example, the total volume sensor 148 may be positioned in the first limb 108 and/or on the first limb 108, in the second limb 110 and/or on the second limb 110, in the housing 118 and/or on the housing 118, or the like.

The shunt device 102 may further include a network interface hardware 137, which may be coupled to or within the pump chamber 104 and/or the first and second limbs 108/110. The network interface hardware 137 may include any wired or wireless networking hardware, such as a modem, a LAN port, a wireless fidelity (Wi-Fi) card, WiMax card, mobile communications hardware, and/or other hardware for communicating with other networks and/or devices. For example, the network interface hardware 137 may provide a communications link between the plurality of sensors 152 and the network 154 along with any other components of the example wireless shunt device monitoring system 101 depicted in FIG. 1. Therefore, the wireless communication between the controller 112, the power device 172 and the shunt device 102 may be provided through the network interface hardware 137.

In other embodiments, each or some of the plurality of sensors 152 may independently transmit data directly from the respective sensor to the controller 112, the power device 172, and the like of the example wireless shunt device monitoring system 101 depicted in FIG. 1. Therefore, the wireless communication between the controller 112, the power device 172 and the shunt device 102 may be provided directly through the respective sensor and not through the network interface hardware 137.

Still referring to FIG. 3A, the shunt device 102 may include a sensor data storage device 135, which may generally be a storage medium, which may contain one or more data repositories for storing data that may be received and/or generated. The sensor data storage device 135 may be any physical storage medium, including, but not limited to, a hard disk drive (HDD), memory, removable storage, and/or the like. While the sensor data storage device 135 is depicted as a local device, it should be understood that the sensor data storage device 135 may be a remote storage device, such as, for example, a server-computing device or the like. Illustrative data that may be contained within the sensor data storage device 135 includes data received from each one of the plurality of sensors 152, as described in greater detail herein. The illustrative data that may be contained within the sensor data storage device 135 may be an overflow of data that cannot be stored or transmitted by the plurality of sensors 152. Further, the illustrative data that may be contained within the sensor data storage device 135 may include data received from each one of the plurality of sensors 152 that may be sent to the controller 112 as a batch of data, or at discrete periods of time.

A local interface 139, such as a bus, wired Ethernet, wireless transceiver(s), and/or the like, may interconnect the various components (e.g., sensor data storage device 135, network interface hardware 137, and the plurality of sensors 152).

Now referring to FIG. 3B, an isolated view of the pump chamber 104 and a portion of each of the first and second limbs 108, 110 is schematically depicted depicting an alternative embodiment to FIG. 3A. In the depicted embodiment, the plurality of sensors 152 are positioned within the first and second limbs 108, 110. That is, the pressure sensor 140, the temperature sensor 142, the glucose sensor 144, the pH sensor 146, the total volume sensor 148, and the oxygen concentration sensor 150 are moved from the pump chamber 104 to the first and second limbs 108, 110. It should be understood that the placement of each of the sensors of the plurality of sensors 152 is for illustrative purposes and any of the sensors may be positioned in the first and/or second limbs 108, 110. It should also be appreciated that the plurality of sensors 152 may be positioned in various combinations between the first and second limbs 108, 110 and the pump chamber 104. That is, a combination of the first and second aspect described between FIGS. 3A-3B are contemplated.

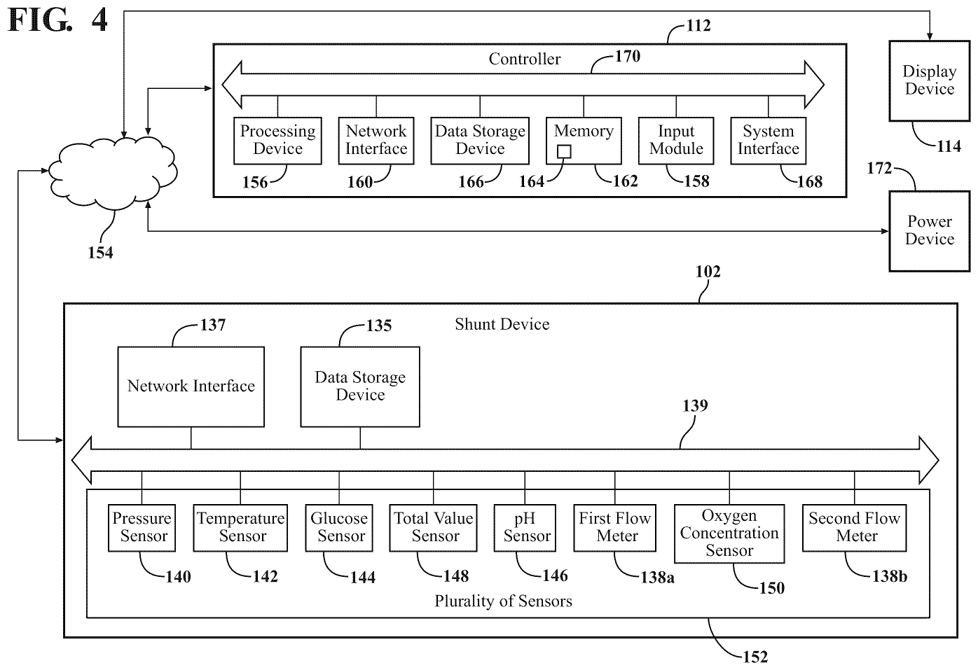

Now referring to FIG. 4, various illustrative internal components of the controller 112, the shunt device 102, the display device 114, and the power device 172 communicatively coupled together according to embodiments is schematically depicted. More specifically, the controller 112 may be communicatively coupled to the internal components of the shunt device 102, the display device 114, and the power device 172 via a network 154. The network 154 may include a wide area network (WAN), such as the Internet, a local area network (LAN), a mobile communications network, a public service telephone network (PSTN), a personal area network (PAN), a metropolitan area network (MAN), a virtual private network (VPN), and/or another network that can electronically connected the controller 112, internal components of the shunt device 102, and the display device 114 together.

The controller 112 may be an electronic control unit that includes a non-transitory computer-readable medium for completing the various processes described herein, embodied as hardware, software, and/or firmware, according to embodiments shown and described herein. While in some embodiments the controller 112 may be configured as a general purpose computer with the requisite hardware, software, and/or firmware, in other embodiments, the controller 112 may also be configured as a special purpose computer designed specifically for performing the functionality described herein. For example, the controller 112 may be a device that is particularly adapted to automatically receive a plurality of data from the plurality of sensors 152 so to determine, interpret and output the data to the display device 114 to display the particular desirable data related to the subject 117, data regarding the movement and/or transporting of fluids 115 (FIGS. 2A-2B) within the shunt device 102, and/or data related to the shunt device 102 itself.

In another example, the controller 112 may be a device that is particularly adapted to utilize the plurality of sensors 152 for the purposes of monitoring various aspects of the physiology and fluids of the subject 117 that may be important to the treating physician in monitoring the function of the shunt device 102 and/or underlying health of the subject 117 such as a flow rate data to monitor for fluid overload, which may potentially trigger Disseminated Intravascular Coagulation (DIC), a total volume throughput data to communicate to the physician how much fluid, such as the fluids 115 (FIGS. 2A-2B) has been removed from the peritoneal cavity 128 and/or pleural cavity 132, pressure data to monitor differential between the first and second limbs 108, 110 to detect a clog forming and may also be used to track compliance for flushing each day to prevent clogs, a temperature data as a marker of possible infection, a glucose data as a marker for possible infection such as septic peritonitis, a pH/oxygenation data as a marker for possible infection such as bacterial peritonitis, protein data in the fluids 115 (FIGS. 2A-2B) data, albumin data in the fluids 115 (FIGS. 2A-2B) data, and/or the like.

In embodiments where the controller 112 is a general purpose computer, the systems and methods described herein provide a mechanism for improving functionality by providing a wireless gathering and transmitting of data from the shunt device 102 to the controller 112, which transforms the data into a visual data, such as within a user interface, that is visually displayed on the display device 114, which is an improvement in the visual display of real time medical data about the subject 117 to the treating physician.

Still referring to FIG. 4, the controller 112 may generally be standalone computing system. In some embodiments, the controller 112 may be a plurality of computing systems (e.g., server computing device(s), user facing computer device(s), and/or the like).

As also illustrated in FIG. 4, the controller 112 may include a processing device 156, an input module 158, network interface hardware 160, a non-transitory memory component 162, a data storage device 166, a system interface 168. A local interface 170, such as a bus or the like, may interconnect the various components.

The processing device 156, such as a computer-processing unit (CPU), may be the central processing unit of the controller 112, performing calculations and logic operations to execute a program. The processing device 156, alone or in conjunction with the other components, is an illustrative processing device, computing device, or combination thereof. The processing device 156 may include any processing component configured to receive and execute instructions (such as from the data storage device 166 and/or the memory component 162).

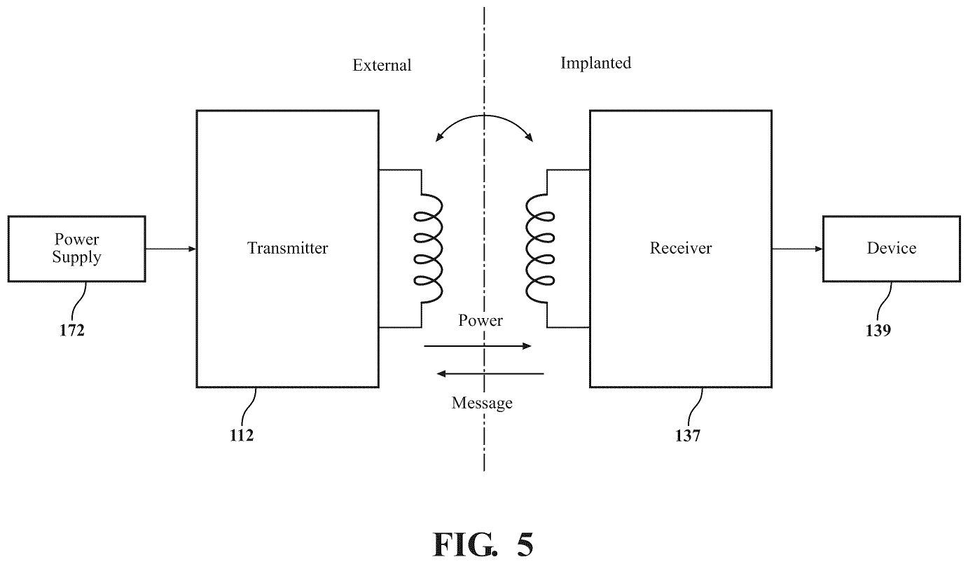

The memory component 162 may be configured as a volatile and/or a nonvolatile computer-readable medium and, as such, may include random access memory (including SRAM, DRAM, and/or other types of random access memory), read only memory (ROM), flash memory, registers, compact discs (CD), digital versatile discs (DVD), and/or other types of storage components. Further, the memory component 162 may be a non-transitory, processor-readable memory. The memory component 162 may include one or more programming instructions thereon that, when executed by the processing device 156, cause the processing device 156 to complete various processes, such as the processes described herein with respect to FIG. 5.

Still referring to FIG. 4, the programming instructions stored on the memory component 162 may be embodied as a plurality of software logic modules 164, where each software logic modules 164 provides programming instructions for completing one or more tasks, as described in greater detail herein. Still referring to FIG. 4, the software logic modules 164 includes a plurality of different pieces of logic, each of which may be embodied as a computer program, firmware, and/or software/hardware, which may be executable by the processing device 156.

The network interface hardware 160 may include any wired or wireless networking hardware, such as a modem, a LAN port, a wireless fidelity (Wi-Fi) card, WiMax card, mobile communications hardware, and/or other hardware for communicating with other networks and/or devices. For example, the network interface hardware 160 may provide a communications link between the display device 114 and the shunt device 102 and any other components of the example wireless shunt device monitoring system 101 depicted in FIG. 1. Therefore, the wireless communication between the controller 112, the shunt device 102 and the display device 114 may be provided through the network interface hardware 160.

Still referring to FIG. 4, the data storage device 166, which may generally be a storage medium, may contain one or more data repositories for storing data that is received and/or generated. The data storage device 166 may be any physical storage medium, including, but not limited to, a hard disk drive (HDD), memory, removable storage, and/or the like. While the data storage device 166 is depicted as a local device, it should be understood that the data storage device 166 may be a remote storage device, such as, for example, a server-computing device or the like. Illustrative data that may be contained within the data storage device 166 includes data received from each one of the plurality of sensors 152, as described in greater detail herein.

Still referring to FIG. 4, the input module 158 may include input hardware (e.g., a keyboard, a mouse, a joystick, a touch screen, a remote control, a pointing device, a video input device, an audio input device, a haptic feedback device, and/or the like) that allows a system administrator and/or physicians to retrieve data, set alarm limits for each of the data set threshold values for each of the data of the plurality of sensors that may be monitored and/or the like for the example wireless shunt device monitoring system 101 (FIG. 1). In some embodiments, the physician may obtain a history of the data stored in the data storage device 166 and/or access and modify set alarm limits for each of the data set threshold values for each of the data of the plurality of sensors that may be monitored in real time to only alert when a particular or combination of data received from the plurality of sensors 152 are below or exceed predetermined thresholds established by the physicians and saved in the data storage device 166, executed by the various logic modules of the memory component 162 and executor by the processing device 156. As such, such instruction changes may prompt the processing device 156 to execute logic stored on the memory component 162.

The system interface 168 may generally provide the controller 112 with an ability to interface with one or more external devices such as, for example, a user-computing device, a server-computing device, and the like. Communication with external devices may occur using various communication ports (not shown). An illustrative communication port may be attached to a communications network.

Still referring to FIG. 4 and now also to FIG. 5, the power device 172, while depicted in FIG. 4 as being a separate component, may be included within the controller 112. The power device 172 may be coupled to the controller 112 and may be configured to provide a power to the sensor data storage device 135, the network interface hardware 137, the plurality of sensors 152, and/or the like. That is, in some embodiments, the sensor data storage device 135, the network interface hardware 137 (e.g., a receiver of power that then powers other components such as the plurality of sensors 152, the sensor data storage device 135, and/or the like), and/or the plurality of sensors 152, from time to time, may require power from the power device 172 to recharge certain devices such a battery, a solid state energy device that provides power to the one or more of the plurality of sensors 152, the sensor data storage device 135, and/or the network interface hardware 137, or directly to one or more of the plurality of sensors 152, the sensor data storage device 135, and/or the network interface hardware 137, each of which may be integrated within each of the plurality of sensors 152 and/or may be separate, independent components communicatively coupled together and may share the common implanted power source (e.g., the network interface hardware 137) configured to receive a wireless charge and distribute that energy to other components (e.g., the plurality of sensors 152, the sensor data storage device 135, and/or the like).

As such, the transmission of power from the power device 172 may be wireless via inductive properties. That is, the power device 172 may be inductively coupled to the one or more of the plurality of sensors 152, the sensor data storage device 135, the network interface hardware 137, and/or a spate and independent power storage device associated therewith, such as housed within or on the shunt device 102. This is non-limiting and the power device 172 may be electrically coupled and supply power to the one or more of the plurality of sensors 152, the sensor data storage device 135, the network interface hardware 137, and/or or a power storage device associated therewith, such as housed within or on the shunt device 102 via resonant inductive coupling, capacitive coupling, electromagnetic radiation, microwaves, light waves via laser beams, and/or the like.

The circuit architecture of FIG. 5 is merely an example of a wireless power transfer that integrates an ultra-low power intelligent power management, an RF to DC converter with very low power sensitivity and high power conversion efficiency (PCE). In some embodiments, the power device 172 may be an active device such as an electric generator, a battery, and/or the like. In other embodiments, the power device 172 may supply power to the one or more of the plurality of sensors 152, the sensor data storage device 135, and/or the network interface hardware 137 via a vibration, a UV/IR energy, solar and wind, radio frequency, and/or the like.

It should be understood that while some of the components of FIG. 4 are illustrated as residing within the controller 112, the display device 114 and/or the shunt device 102, this is merely an example thereof. In some embodiments, one or more of the components may reside solely within controller 112, or, in the alternative, one or more components may be external to the shunt device 102, the display device 114, and to the controller 112. For example, the power device 172 may reside within the controller 112.

It should also be appreciated that the controller 112 and/or the shunt device 102 may receive new and/or updated instructions or configurations as needed. For example, changing of threshold levels and/or alarm settings in the controller 112. Another example would be a different or improved sensor of the plurality of sensor and the data detect and transmitted therefrom. It should also be appreciated that the software logic module 164, the memory component 162 and/or the processing device 156 may also receive updates and/or new user initiated programs from time to time. These updates may be based on the type of sensors of the plurality of sensors 152, the threshold levels for alerting the physician, and/or the like. Moreover, the subject 117, or a remote third party, such as a physician, may use an application installed on a smart device, tablet, wearable, or a computer that communicates with the controller 112. Such devices may be used to update the operating parameters, alerts, settings, and/or the like.

In embodiments, the controller 112 may monitor the data provided by the plurality of sensors 152 for a plurality of predetermined parameters and metrics, such as flow rate data to monitor for fluids 115 (FIGS. 2A-2B) overload, which may potentially trigger Disseminated Intravascular Coagulation (DIC), a total volume throughput data to communicate to the physician how much fluid, such as the fluids 115 (FIGS. 2A-2B) has been removed from the peritoneal cavity 128 and/or pleural cavity 132, pressure data to monitor differential between the first and second limbs 108, 110 to detect a clog forming and may also be used to track compliance for flushing each day to prevent clogs, temperature data as a marker of possible infection, glucose data as a marker for possible infection such as septic peritonitis, pH/oxygenation data as a marker for possible infection such as bacterial peritonitis, protein in the fluids 115 (FIGS. 2A-2B) data, albumin in the fluids 115 (FIGS. 2A-2B) data, and/or the like. Should any of these metrics fall below or above a certain threshold, the subject 117 (FIGS. 2A-2B) may be alerted to perform a manual pump on the pump chamber 104 (FIG. 1), the physician may be alerted for further diagnosis, and/or the like.

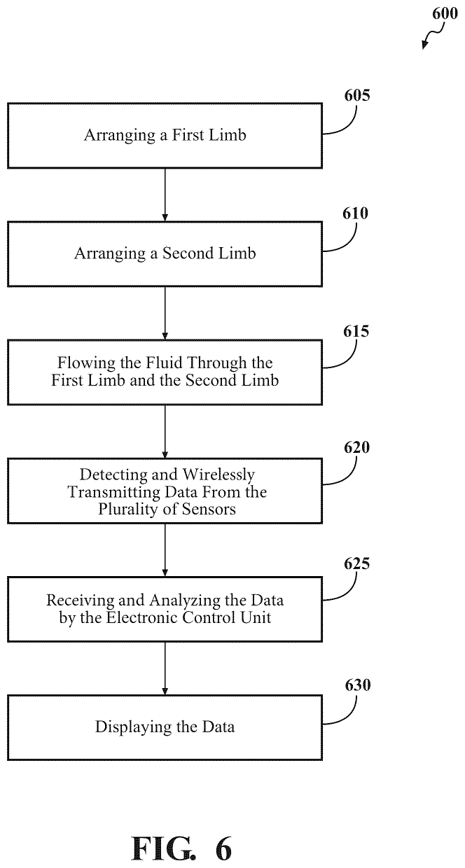

Referring now to the flow diagram of FIG. 6 that graphically depicts an illustrative method 600 in conjunction with FIGS. 1 and 2A-2B of moving the fluids 115 from the peritoneal cavity 128 to the venous system 130 or from the pleural cavity 132 to the peritoneum 134 (peritoneal cavity) of the subject 117 is schematically depicted. Although the steps associated with the blocks of FIG. 6 will be described as being separate tasks, in other embodiments, the blocks may be combined or omitted. Further, while the steps associated with the blocks of FIG. 6 will described as being performed in a particular order, in other embodiments, the steps may be performed in a different order. Further, the depiction of FIG. 6 and the accompanying description below is not meant to limit the subject matter described herein or represent an exact description of how the shunt device 102 operates, but instead is meant to provide a simple schematic overview to illustrate the general operation of the shunt device 102 of the method described herein.

At block 605, a first limb 108 may be arranged within the peritoneal cavity 128 or within the pleural cavity 132 of the subject 117. As illustrated in FIGS. 2A-2B, the first limb 108 includes a tip portion 124, which may be arranged within the peritoneal cavity 128 or the pleural cavity 132 of the subject 117. The first limb 108 fluidly couples the remaining components of the shunt device 102 to the either the peritoneal cavity 128 or the pleural cavity 132.

At block 610, a second limb 110 may be arranged within either the venous system 130 or the peritoneum (peritoneal cavity) 134 depending on whether the first limb 108 may be arranged within the peritoneal cavity 128 or within the pleural cavity 132, respectively. As illustrated in FIGS. 2A-2B, the second limb 110 include the tip portion 122 and may be arranged within either the venous system 130 or the peritoneum (peritoneal cavity) 134 of the subject 117. The first limb 108 and the second limb 110 are fluidly coupled to one another via the pump chamber 104, which create the shunt device 102.

At block 615, fluid may be flowed from the either the peritoneal cavity 128 to the venous system 130 or from the pleural cavity 132 to the peritoneum (peritoneal cavity) 134 of the subject 117. With reference to FIG. 2A, the first limb 108 and the second limb 110 fluidly couple the peritoneal cavity 128 to the venous system 130 of the subject 117. The fluids 115, which may be present in the peritoneal cavity 128, flows through the first limb 108, the pump chamber 104 and the second limb 110 due to a pressure differential between the peritoneal cavity 128 to the venous system 130. For example, the pressure differential may be created by the subject 117 displacing the flexible membrane 120 of the pump chamber 104, which increases pressure within their abdominal cavity. The fluids 115 flows continuously through the first limb 108, into and through the second limb 110 in order to continuously drain the peritoneal cavity 128.

With reference to FIG. 2B, the first limb 108 and the second limb 110 fluidly couple the pleural cavity 132 to the peritoneum (peritoneal cavity) 134 of the subject 117. The fluids 115, which may be present in the pleural cavity 132, flows through the first limb 108, the pump chamber 104 and the second limb 110 due to a pressure differential between the peritoneal cavity 128 to the peritoneum (peritoneal cavity) 134. The pressure differential may be created by the subject 117 displacing the flexible membrane 120 of the pump chamber 104 increasing pressure within the pleural cavity 132. The fluids 115 flows continuously through the first limb 108, into and through the second limb 110 in order to continuously drain the pleural cavity 132.

Now referring back to FIG. 6, and with reference to FIGS. 2A-2B, 3A-3B and 4, at block 620, the plurality of sensors 152 detect and transmit data wirelessly to the controller 112. The plurality of sensors 152 detect and/or sense and transmit (e.g., wirelessly) a plurality of data to the controller 112 for the purposes of monitoring various aspects of the physiology and fluids of the subject 117 that may be desired in monitoring the function of the shunt device 102 and/or underlying health of the subject 117. Example data may be flow rate data to monitor for fluid overload, which may potentially trigger Disseminated Intravascular Coagulation (DIC), total volume throughput data to communicate to the physician how much fluid (e.g., ascites, effusion, and the like) has been removed from the peritoneal cavity 128 and/or pleural cavity 132, pressure data to monitor differential between the first and second limbs 108, 110 to detect a clog forming and may also be used to track compliance for flushing each day to prevent clogs, a temperature data as a marker of possible infection, glucose data as a marker for possible infection such as septic peritonitis, pH/oxygenation data as a marker for possible infection such as bacterial peritonitis, protein in the fluids 115 (FIGS. 2A-2B) data, albumin in the fluids 115 (FIGS. 2A-2B) data, and/or the like.

At block 625, the controller 112 analyzes the received data from one or more of the plurality of sensors 152. Based on the received data and the plurality of predetermined parameters and metrics, the data may be displayed on a display device 114, at block 630, and based on the various thresholds, the controller 112 may output an alert, such as an aural (via a speaker), visual (via a display), or other type alert.

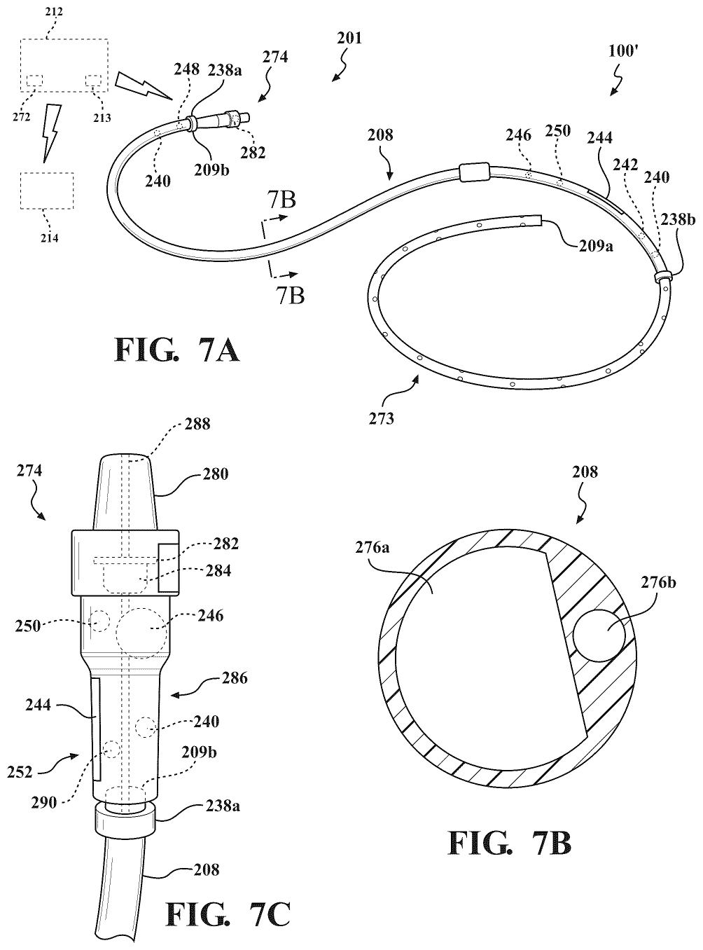

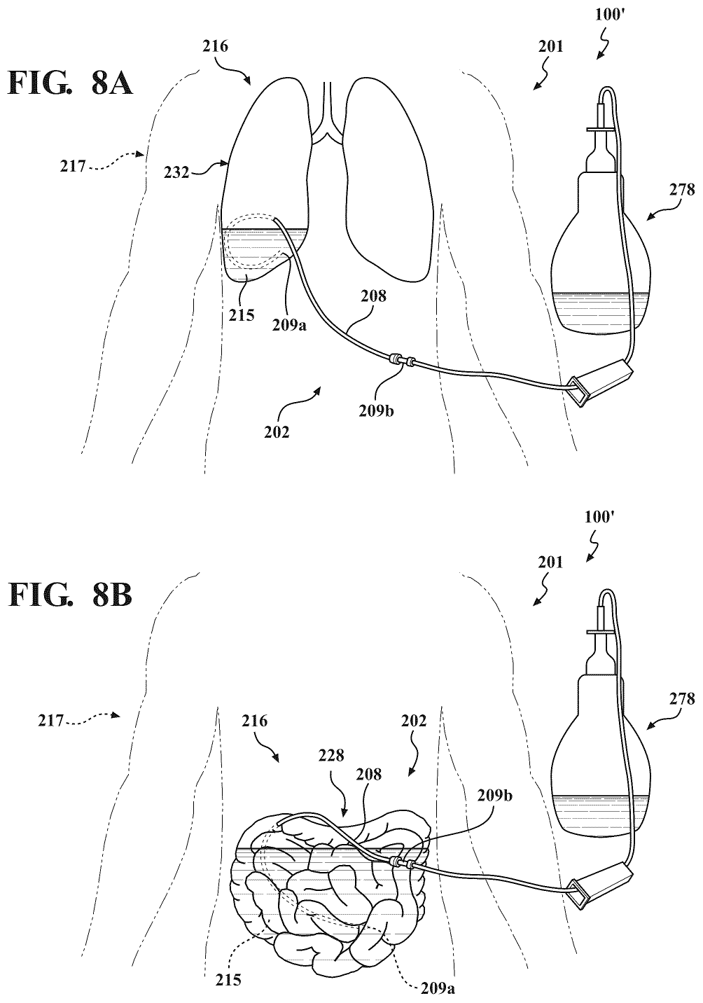

Referring now to FIGS. 7A-7C and 8A-8B, another embodiment of the device monitoring system 100′ is generally depicted. In this aspect, the device monitoring system 100′ is a catheter device monitoring system 201 that may be wired or wireless, as discussed in greater detail herein. It is understood that the example catheter device monitoring system 201 is similar to the example wireless shunt device monitoring system 101 with the exceptions of the features described herein. As such, like features will use the same reference numerals with a prefix “2” for the reference numbers. As such, for brevity reasons, these features will not be described again.

As illustrated, the catheter device monitoring system 201 may include a catheter device 202 including a catheter 208, which includes a distal end 209a and an opposite proximal end 209b. Further, a plurality of perforations 273, or bores, or openings, may be positioned near or extending a predetermined length from the distal end 209a towards the proximal end 209b. The catheter device monitoring system 201, in some embodiments, may further include an external valve portion 274. That is, in the illustrative embodiment of FIG. 7A, the catheter device monitoring system 201 is schematically illustrated as including external valve portion 274. In other embodiments, the external valve portion 274 is not included. For example, as illustrated in FIGS. 8A-8B, the catheter device monitoring system 201 is illustrated without the external valve portion 274. Further, the catheter device monitoring system 201 may further include a controller 212, which may include an electronic control unit 213, a display device 214, and a power device 272, though a greater or fewer number of components are contemplated and possible. Further, each of the controller 212, the electronic control unit 213, and the power device 272 may be separate or independent components.

As will be described in greater detail herein, the catheter device 202 may be configured, as illustrated in FIG. 8A, as an indwelling pleural catheter device to transfer fluids 215 from a pleural cavity 232 to the external fluid retention device 278 thereby palliating the symptoms associated with recurrent pleural effusion. The catheter device 202 may also be configured, as illustrated in FIG. 8B, as an indwelling peritoneal catheter device to passively move fluids 215 from a peritoneal cavity 228 to an external fluid retention device 278 thereby palliating the symptoms associated with recurrent ascites.

It is noted that the catheter device 202 may be used to move or transport any type of fluid from and to any type of cavity/area within a body 216 of a subject 217. For example, the catheter device 202 may be used to move or transport ascites from the bladder to outside or external to the body 217.

Referring now to FIG. 7B, a cross section of the catheter taken from line 7-7 is schematically depicted. As illustrated, in some embodiments, the catheter 208 of the catheter device 202 may include a pair of lumens 276a, 276b extending a length of the catheter 208. In embodiments, one lumen 276a may be configured to receive and transport fluid as a drainage pathway and the other lumen 276b may provide a pathway for electronics, such as wires and the like, as discussed in greater detail herein. In some embodiments, the lumen 276a of the pair of lumens 276a, 276b has a larger diameter or size than the other lumen 276b of the pair of lumens 276a, 276b. In other embodiments, the pair of lumens 276a, 276b have identical diameters or sizes. Further, in some embodiments, the lumen 276b of the pair of lumens 276a, 276b may generally be circular in shape while the lumen 276a of the pair of lumens 276a, 276b includes a flat portion to form a substantially half moon or D-shape. These shapes are non-limiting and each of the pair of lumens 276a, 276b may be any shape including square, rectangular, hexagonal, octagonal, and the like.

As such, either the external valve portion 274, the catheter 208, or both generally provide the structure to which the various components are connected. The external valve portion 274 and/or the catheter 208 may be flexible or rigid. Further, in some embodiments, portions of the catheter 208 may seal components, such as a wires, motor, control unit, batteries, and the like, from the outside environment, such as inside of the subject's body 216. In this embodiment, the catheter 208 and/or the external valve portion 274 may be made from a non-conductive material to allow for wireless communication with the control unit and may allow for induction charging of the battery and/or allow wires to pass therethrough.

Referring now to FIG. 8A, the catheter device 202 may be an indwelling pleural catheter device that passively moves fluids 215 from the pleural cavity 232 to the external fluid retention device 278 thereby palliating the symptoms associated with recurrent pleural effusion. That is, portions of the catheter device 202 (e.g., the distal end 209a and portions of the catheter 208) may be arranged within the body 216 of a subject 217 in order to move or transport the fluids 215 from the pleural cavity 232 to the external fluid retention device 278. The catheter 208 may be arranged within the pleural cavity 232 in such a way that the fluids 215 may enter the distal end 209a of the catheter 208. For example, the distal end 209a of the catheter 208 may be arranged in the lower section of the pleural cavity 232 where the fluids 215 collect within the pleural cavity 232. The catheter 208 may extend through an incision within the wall of the pleural cavity 232.

Still referring still to FIG. 8A, the catheter 208 may be arranged such that the fluids 215 may be transported from the pleural cavity 132 to the external fluid retention device 278 through the catheter device 202, the external fluid retention device 278 accepts and diffuses the fluids 215 through various techniques, such as negative pressure, and the like. As noted above, the at external valve portion 274 may prevent backflow of the fluid from the external fluid retention device 278 to the proximal end 209b.

Due to the arrangement of the catheter device 202 within the body 216 of the subject 217, with the distal end 209a of the catheter 208 arranged within the pleural cavity 232 and the proximal end 209b positioned outside of the body 216 and in fluid communication with the external fluid retention device 278, a passive pressure-driven flow of fluids 215 may be formed between the external fluid retention device 278 and the pleural cavity 232 through the catheter device 202. Since the external fluid retention device 278 and the pleural cavity 232 are fluidly coupled, any pressure gradient between the external fluid retention device 278 and the pleural cavity 232 will create a pressure-driven flow of fluids 215. For example, as pressure increases within the pleural cavity 232, the fluids 215 stored within the pleural cavity 232 will be forced through the catheter 208 as a pressure release for the pleural cavity 232. The pressure within the pleural cavity 232 may be increased by having the subject 217 exert their diaphragm into their abdominal cavity, or by breathing normally. Since the action of breathing or exerting a diaphragm does not increase the pressure within the pleural cavity 232, a pressure gradient may be formed between the pleural cavity 232 and the pleural cavity 232, with the high pressure being present in the pleural cavity 232, and the low pressure being present in the external fluid retention device 278. This difference in pressures passively flows the fluids 215 from the pleural cavity 232 to the external fluid retention device 278, without the need of additional pumping mechanisms.

Referring to FIG. 8B, the catheter device 202 may be an indwelling peritoneal catheter device that passively moves fluids 215 from the peritoneal cavity 228 or source to the external fluid retention device 278 thereby palliating the symptoms associated with recurrent ascites. That is, the catheter device 202 may be at least partially implanted (e.g., at least the distal end 209a) within the body 216 of a subject 217 in order to move or transport the fluids 215 from the peritoneal cavity 228 to the external fluid retention device 278. Specifically, the distal end 209a and portions of the catheter 208 may be arranged within the peritoneal cavity 228 of the body 216 of the subject 217. However, in embodiments, the catheter 208 may be arranged in any area of the body 216, which requires a fluid, such as ascites fluids, to be moved or transported. The catheter 208 may be arranged within the peritoneal cavity 228 in such a way that the fluids 215 may enter the catheter 208 via the distal end 209a. For example, the distal end 209a of the catheter 208 may be arranged in the lower section of the peritoneal cavity 228 where the fluids 215 collects within the peritoneal cavity 228. The catheter 208 may extend through an incision within the wall of the peritoneal cavity 228.

Still referring still to FIG. 8B, the fluids 215 may be transported from the peritoneal cavity 128 to the external fluid retention device 278 through the catheter device 202, such that the external fluid retention device 278 accepts and diffuses the fluids 215 through various techniques, such as negative pressure, and the like. Since the peritoneal cavity 228 and the external fluid retention device 278 are fluidly coupled, any pressure gradient between the peritoneal cavity 228 and the external fluid retention device 278 will create a pressure-driven flow of fluid. For example, as pressure increases within the peritoneal cavity 228, the fluid stored within the peritoneal cavity 228 may be forced through the catheter 208 as a pressure release for the peritoneal cavity 228. The pressure within the peritoneal cavity 228 may be increased by having the subject exert their diaphragm into their abdominal cavity, or by breathing normally. Since the action of breathing or exerting a diaphragm does not increase the pressure within the external fluid retention device 278, a pressure gradient may be formed between the peritoneal cavity 228 and the external fluid retention device 278, with the high pressure being present in the peritoneal cavity 228, and the low pressure being present in the external fluid retention device 278. This difference in pressures passively flows the fluids 215 from the peritoneal cavity 228 to the external fluid retention device 278, without the need of additional pumping mechanisms. As noted above, in embodiments that include the external valve portion 274, the external valve portion 274 may prevent backflow of the fluid from the external fluid retention device 278 to the proximal end 209b.

It should be noted that the catheter 208 is not limited to the indwelling peritoneal catheter device or indwelling pleural catheter device and instead may be used in a plurality of additional applications. For example, and without limitation, the catheter 208 may be used as or for percutaneous drainage, post-surgical wound drains, chest tubes, urinary drainage, pericardial effusion drainage, dialysis, external ventricular drains, cerebro-spinal fluid drains, central venous catheters, ports, feeding tubes, peripherally inserted central catheters, and the like. Further, the catheter 208 is not limited to moving ascites or effusion fluids and may instead be used to move or transport any type of fluid from, and to, any type of cavity/area within a body 216 of the subject 217, such as those fluids discussed above with respect to the applications. For example, the catheter 208 may be used to move or transport fluids such as pericardial effusions pus, cerebro-spinal fluid, bile, urine, and the like.

Now referring back to FIG. 7A, in one embodiment of the example catheter device monitoring system 201, the catheter 208 further includes one or more sensors such as a plurality of sensors 252. The plurality of sensors 252 may include a number of various sensors that will be described in greater detail herein.

For example, the catheter 208 may each include the flow meters 238a, 238b. Each of the flow meters 238a, 238b may be strategically positioned near the distal end 209a and the proximal end 209b. Each of the flow meters 238a, 238b may be communicatively coupled to the controller 212 such as via any wireless communication protocol (e.g., Bluetooth, cellular, or similar technology) or may be wired with the electrical components extending the in lumen 276b of the catheter 208. Data gathered by the flow meters 238a, 238b may be gathered and transmitted to the controller 212. As such, the controller 212 may analyze the data received from the each of the flow meters 238a, 238b to determine or calculate a differential flow rate between the fluid received in the distal end 209a and the fluid expelled at the proximal end 209b. A variance in the differential flow rate may indicate potential clogging within the catheter device 202. It should be appreciated that the flow meters 238a, 238b need not be positioned within the catheter 208 and instead may be positioned to gather data from the fluid in contact with the catheter 208 regardless of the fluid flow through the catheter 208 or a fluid flow that surrounds the catheter 208.

Example flow meters 238a, 238b may include implantable flow sensors. Implantable flow sensors may be used to monitor the flow rate of fluids 215 (FIGS. 8A-8B) through tubular walls such within the lumen 276a of the catheter 208. In some embodiments, implantable flow sensors may be fitted around the tubular wall of the catheter 208 external to the flow path. Further, implantable flow sensors may use electromagnetic, ultrasonic, and time of flight techniques where the electromagnetic sensors generate a magnetic field perpendicular to the flow of the fluids 215 (FIGS. 8A-8B) through the lumen 276a of the catheter 208 such that charged ions in the fluids 215 (FIGS. 8A-8B) pass through the magnetic field causing variation in the conductivity as measured by an electrode.

As discussed in greater detail herein, the catheter 208 may also include an additional one or more of the plurality of sensors 252. For example, the catheter 208 may include pressure sensors 240, a temperature sensor 242, a glucose sensor 244, a pH sensor 246, a total volume sensor 248, an oxygen concentration sensor 250, the TENG Film 282, or the like. The flow meters 238a, 238b, the pressure sensors 240, the temperature sensor 242, the glucose sensor 244, the pH sensor 246, the total volume sensor 248, and the oxygen concentration sensor 250, and/or the TENG Film 282 collectively, may be referred to as the plurality of sensors 252. In embodiments, each of the pressure sensors 240, the temperature sensor 242, the glucose sensor 244, the pH sensor 246, the total volume sensor 248, the oxygen concentration sensor 250, and/or the TENG Film 282 may be positioned within or around the catheter 208 and may each be wired to utilize the lumen 276b (FIG. 7B) of the catheter 208 and/or each may be wireless utilizing the wireless communication protocols between the control unit and the plurality of sensors 252 discussed in greater detail herein.

The pressure sensors 240 may include a pair of sensors and may be any sensor configured to output a signal indicative of a pressure within the catheter 208. For example, one of the pressure sensors 240 may be positioned near the distal end 209a and the other near the proximal end 209b to output the pressure of the fluid that may be used as a differential calculation by the controller 212. As an example and without limitation, the pressure sensors 240 may use a left ventricular assist device (LVAD) technology to detect and either wired or wirelessly transmit related to the perfusion of the fluids 215 (FIGS. 8A-8B) within the catheter 208. In other embodiments, each of the pressure sensors 240 may be a potentiometric type pressure sensors, inductive type pressure sensors, capacitive type pressure sensors, piezoelectric type pressure sensors, strain gauge type pressure sensors, variable reluctance type pressure sensors, and/or the like.

It should be appreciated that the pressure sensors 240 are not limited to be positioned within the catheter 208 or output a signal indicative of a pressure within the lumen 276a of the catheter 208. The pressure sensors 240 may be positioned anywhere to gather data from the fluid in contact with the catheter 208 regardless of the fluid flowing through the lumen 276a or fluid flow that surrounds the catheter 208 external to the catheter 208. For example, the pressure sensors 240 may be positioned in either of the lumens 276a, 276b and/or on catheter 208, or the like.

The temperature sensor 242 may include any sensor configured to output a signal indicative of a temperature of a fluid within the lumen 276a of the catheter 208. For example, the temperature sensor 242 may be a wired or wireless fibre optic temperature probe that detects and transmits data related to the temperature of the fluids 215 (FIGS. 8A-8B) moving through the lumen 276a of the catheter 208. In other embodiments, the temperature sensor 242 may a thermocouple type sensor, a resistance type temperature detector, semiconductor type temperature sensors, and/or the like.

It should be appreciated that the temperature sensor 242 need not be positioned within the catheter 208 (e.g., within either of the lumens 276a, 276b) and instead may be positioned to gather data from the fluid in contact with the catheter 208 regardless of the fluid flowing through the catheter 208 and/or a fluid flow that surrounds portions of the catheter 208. For example, the temperature sensor 242 may be positioned in either of the lumens 276a, 276b and/or on catheter 208, or the like.

The glucose sensor 244 may include any sensor configured to output a signal indicative of a glucose level within a fluid within the lumen 276a of the catheter 208. For example, the glucose sensor 244 may be an implantable type sensor, such as those that include a substrate with a sensor, an RF detection circuit, a glucose affinity polymer and an antenna. As such, the glucose sensor 224 detects and either wired or wirelessly transmits data related to glucose in the fluids 215 (FIGS. 8A-8B) moving through the lumen 276a if the catheter 208. The glucose sensor may use different techniques of glucose recovery such as reverse iontophoresis, direct subcutaneous implantation and microdialysis.

It should be appreciated that the glucose sensor 244 need not be positioned within the lumen 276a of the catheter 208 and instead may be positioned to gather data from the fluid in contact with the catheter 208 regardless of the fluid flowing through the lumen 276a of the catheter 208 and/or the fluid flow that surrounds the catheter 208 external to the catheter 208. For example, the glucose sensor 244 may be positioned in either of the lumens 276a, 276b and/or on catheter 208, or the like.

The pH sensor 246 and the oxygen concentration sensor 250 may include any sensor or multiple sensors configured to output a signal indicative of an oxygen concentration and/or pH level within a fluid within the lumen 276a of the catheter 208. For example, the pH sensor 246 and the oxygen concentration sensor 250 may be included in a single sensor assembly or may be independent sensors. The pH sensor 246 and/or the oxygen concentration sensor 250 detect and either wired or wirelessly transmit data related to the pH and oxygen concentration, respectively, of the fluids 215 (FIGS. 8A-8B) moving through the lumen 276a of the catheter 208. In some embodiments, combination sensors may use amperometric, potentiometric and/or impedance sensors for measurement of the oxygen concentration, the pH value and the impedance in the fluids 215 (FIGS. 8A-8B) moving through the lumen 276a of the catheter 208. The various electronic circuits are designed for maximum energy efficiency to allow, together with the high integration density, as small as possible and wireless active implants.

It should be appreciated that the pH sensor 246 and the oxygen concentration sensor 250 need not be positioned within the lumen 276a of the catheter 208 and instead may be positioned to gather data from the fluid in contact with the lumen 276a of the catheter 208 regardless of the fluid flowing through the lumen 276a if the catheter 208 and/or the fluid flow that surrounds the catheter 208 external to the catheter 208. For example, the pH sensor 246 may be positioned in either of the lumens 276a, 276b and/or on catheter 208, or the like.

The total volume sensor 248 may include any sensor configured to output a signal indicative of a total volume of fluid passing through the lumen 276a of the catheter 208, and either wired or wirelessly transmit data related to total volume of fluids 215 (FIGS. 8A-8B) moving through the lumen 276a of the catheter 208. As such, the total volume sensor 248 may assist the controller 212 in detecting undesirable conditions such as increased ascites fluids in various cavities. In some embodiments, the total volume sensor 248 may be ultrasonic type flow sensors, micromachined type flow sensors, membrane-less mass flow micro type sensors, carbon nanotubes type for sensing applications, thermal type flow sensors, MEMS type flow sensors, digital type sensors for fluids 215 (FIGS. 8A-8B) flow, vortex-shedding type sensors, and/or the like.

It should be appreciated that the total volume sensor 248 need not be positioned within the lumen 276a of the catheter 208 and instead may be positioned to gather data from the fluid in contact with the lumen 276a of the catheter 208 regardless of the fluid flowing through the lumen 276a of the catheter 208 and/or the fluid flow that surrounds the catheter 208 external to the catheter 208. For example, the total volume sensor 248 may be positioned in either of the lumens 276a, 276b and/or on catheter 208, or the like.

A local interface 239, such as a bus, wired Ethernet, other wired connections, wireless transceiver(s), and/or the like, may interconnect the various components (e.g., sensor data storage device 235, network interface hardware 237, and the plurality of sensors 252).

It should be understood that the placement of each of the sensors of the plurality of sensors 252 is for illustrative purposes and any of the sensors may be positioned anywhere within or around the catheter 208.

Now referring back to FIG. 7C, in another embodiment of the example catheter device monitoring system 201, the external valve portion 274 may further include one or more sensors of the plurality of sensors 252. That is, FIG. 7B is a schematic depiction of an isolated view of the external valve portion 274 depicting an alternative embodiment to FIG. 7A.

In some embodiments, the external valve portion 274 includes a valve top 280, the triboelectric nanogenerator (TENG) film 282 (to replace a disk valve in conventional valve portions), a duckbill valve 284 and a valve body 286. The TENG Film 282 may be referred to as part of or included within the plurality of sensors 252, discussed in greater in detail herein, and may be configured to measure a number of valve accesses as well as may be used as an energy generator (e.g., a power source) for the plurality of sensors 252. The number of valve accesses may include the number of times a mating accessory is connected to the external valve portion 274 to monitor a number of access events. As such, the number of access events may be monitored and compared to a predetermined number of accesses before the potential for an undesirable condition occurs, such as failure, and alert the user when the predetermined number of accesses events has exceeded. Such an alert may warn users to replace the external valve portion 274 before an occurrence of the undesirable conditions.

Further, the external valve portion 274 may include a channel 288 extending from the valve top 280 to the valve body 286 and corresponds to the lumen 276b of the catheter 208 to allow for wired components and electronics to pass through the external valve portion 274, from the catheter 208 to the controller 212 (FIG. 7A).

Further, in some embodiments, the plurality of sensors 252 positioned within the external valve portion 274 may further include an object detection sensor 290. The object detection sensor 290 may include any sensor configured to output a signal indicative of a detection of a presence of any foreign object or debris within the external valve portion 274. For example, the object detection sensor 290 may be a Hall effect sensor, inductive sensor, infrared, and the like to detect and either wired or wirelessly transmit data related to the foreign objects of the fluids 215 (FIGS. 8A-8B) within the external valve portion 274. It should be appreciated that the object detection sensor 290 is not limited to be positioned within the external valve portion 274 or output a signal indicative of foreign objects within the external valve portion 274. Example foreign objects may include, without limitation, encrustation by mineral salts, blood, and other foreign objects not typically found in the fluids 215 (FIGS. 8A-8B).

The external valve portion 274 may be positioned to extend from the proximal end 209b of the catheter 208 and operable to prevent back-flow of any fluids 215 (FIGS. 8A-8B) passing through external valve portion 274 from flowing back toward an originating site of the fluids 215. Further, the duckbill valve 284 is non-limiting and the valve may be a mitre valve, or another type of one-way valve which only allows fluids 215 (FIGS. 8A-8B) to flow in a single direction when a pressure differential is created on opposite sides of the external valve portion 274. Further, there may be more than one external valve portion 274 included in the example catheter device monitoring system 201. It is noted that the external valve portion 274 may include a greater or fewer number of components without departing from the scope of the present disclosure.

Further, in the depicted embodiment of FIG. 7C, one or more of the plurality of sensors 252 are positioned within external valve portion 274. For example, and non-limiting, the pressure sensors 240, the glucose sensor 244, the pH sensor 246, the oxygen concentration sensor 250, and/or one of the flow meters 238b are moved from the catheter 208 and are instead positioned within or otherwise installed onto, positioned in contact with, or coupled to the external valve portion 274. It should be understood that the placement of each of the sensors of the plurality of sensors 252 is for illustrative purposes and any of the sensors may be positioned anywhere on, within, and/or adjacent to the external valve portion 274. It should also be appreciated that the plurality of sensors 152 may be positioned in various combinations between the catheter 208 and the external valve portion 274 first and second limbs 108, 110 and the pump chamber 104. That is, a combination of the first and second aspect described between FIGS. 7A and 7B are contemplated.

Further, the arrangement of at least some of the plurality of sensors 252 positioned within the external valve portion 274 may reduce biocompatibility constraints and allow for larger sized sensors to be positioned within the external valve portion 274 while maintaining direct contact with the fluid. That is, because at least some of the plurality of sensors 252 are positioned external to the body 216 during use, larger sized sensors may be used.

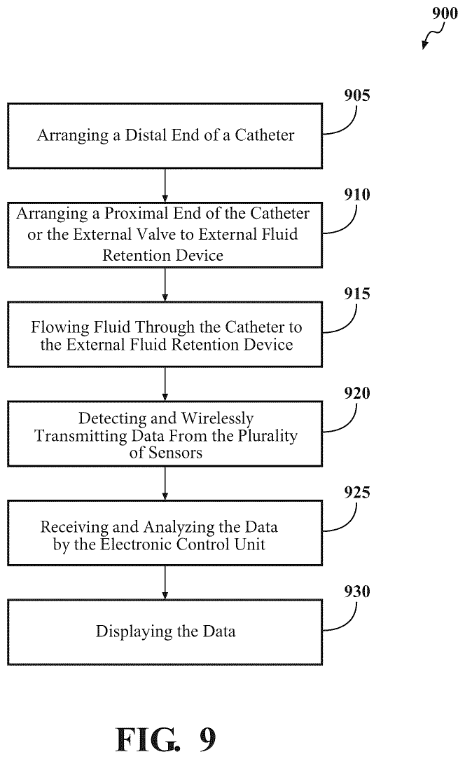

Referring now to the flow diagram of FIG. 9 that graphically depicts an illustrative method 900 in conjunction with FIGS. 7A-7C and 8A-8B of moving the fluids 215 from the peritoneal cavity 228 to the external fluid retention device or from the pleural cavity 232 to the external fluid retention device is schematically depicted. Although the steps associated with the blocks of FIG. 9 will be described as being separate tasks or steps, in other embodiments, the blocks may be combined or omitted. Further, while the steps associated with the blocks of FIG. 9 will described as being performed in a particular order, in other embodiments, the steps may be performed in a different order. Further, the depiction of FIG. 9 and the accompanying description below is not meant to limit the subject matter described herein or represent an exact description of how the catheter device 202 operates, but instead is meant to provide a simple schematic overview to illustrate the general operation of the catheter device 202 of the method described herein.

At block 905, a distal end 209a of the catheter 208 may be arranged within the peritoneal cavity 228 or within the pleural cavity 232 of the subject 217. As illustrated in FIGS. 8A-8B, the distal end 209a and at least a portion of the perforations 273 may be arranged within the peritoneal cavity 228 or the pleural cavity 232 of the subject 217. The catheter 208 fluidly couples the remaining components of the catheter device 202 to the external fluid retention device 278.

At block 910, a proximal end 209b or the external valve 274 may be arranged to fluidly couple to the external fluid retention device 278. At block 915, fluid may be flowed from the either the peritoneal cavity 228 to the external fluid retention device 278 (e.g., through the proximal end 209b directly to the external fluid retention device 278 or through the proximal end 209b into the external valve 274 and into the external fluid retention device 278 either directly or with additional tubing fluidly coupled between the external valve 274 and the external fluid retention device 278) or from the pleural cavity 232 to the external fluid retention device 278 (e.g., through the proximal end 209b directly to the external fluid retention device 278 or through the proximal end 209b into the external valve 274 and into the external fluid retention device 278 either directly or with additional tubing fluidly coupled between the external valve 274 and the external fluid retention device 278). With reference to FIG. 8B, the fluids 215, which may be present in the peritoneal cavity 228, flows through the catheter 208 to the external fluid retention device 278 due to a pressure differential between the peritoneal cavity 228 and the external fluid retention device 278. The fluids 215 may flow continuously through the catheter 208 in order to continuously drain the peritoneal cavity 228.

With reference to FIG. 8A, the catheter 208 fluidly couples the pleural cavity 232 external fluid retention device 278. The fluids 215, which may be present in the pleural cavity 232, flows through the catheter 208 due to a pressure differential between the peritoneal cavity 228 to the external fluid retention device 278. The fluids 215 may flow continuously through the catheter 208 and into external fluid retention device 278 in order to continuously drain the pleural cavity 232.

Now referring back to FIG. 9, and with reference to FIGS. 7A-7C and 8A-8B, at block 920, the plurality of sensors 252 detect and transmit data either wirelessly or via wired connections to the controller 212. The plurality of sensors 252 detect and/or sense and wirelessly, or wired, transmit a plurality of data to the controller 212 for the purposes of monitoring various aspects of the physiology and fluids of the subject 217 that may be desired in monitoring the function of the catheter device 202 and/or underlying health of the subject 217. Example data may be flow rate data to monitor for fluid overload, which may potentially trigger Disseminated Intravascular Coagulation (DIC), total volume throughput data to communicate to the physician how much ascites fluids has been removed from the peritoneal cavity 228 and/or pleural cavity 232, pressure data to monitor differential between the distal end 209a and the proximal end 209b to detect a clog forming and may also be used to track compliance for flushing each day to prevent clogs, temperature data as a marker of possible infection, glucose data as a marker for possible infection such as septic peritonitis, a pH/oxygenation data as a marker for possible infection such as bacterial peritonitis, protein data in the fluids 215 data, albumin data in the fluids 215 data, and/or the like.

At block 925, the controller 212 analyzes the received data from one or more of the plurality of sensors 252. Based on the received data and the plurality of predetermined parameters and metrics, the data may be displayed on a display device 214, at block 930, and based on the various thresholds, the controller 212 may output an alert. It is noted that the plurality of predetermined parameters and metrics may be user-defined or pre-defined based on known medical standards, for any of the received data from one or more of the plurality of sensors 252.

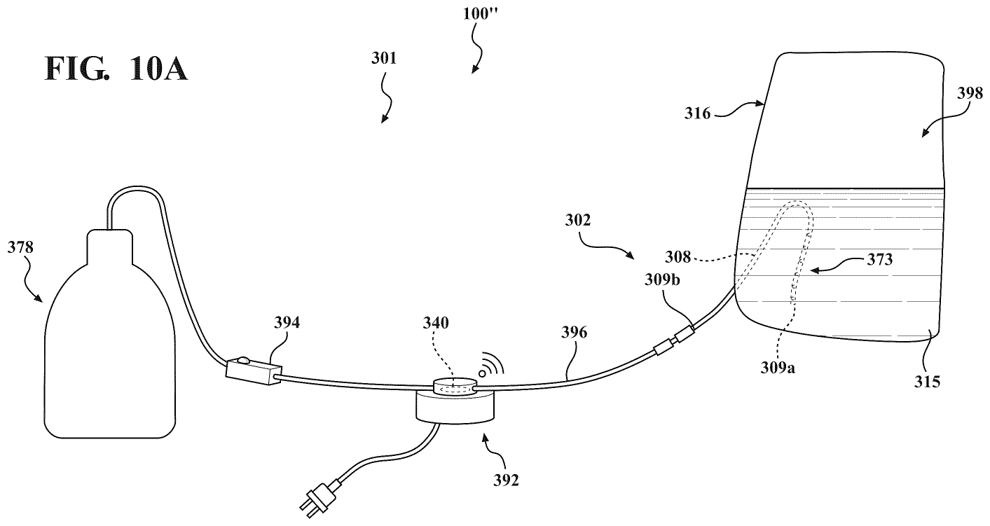

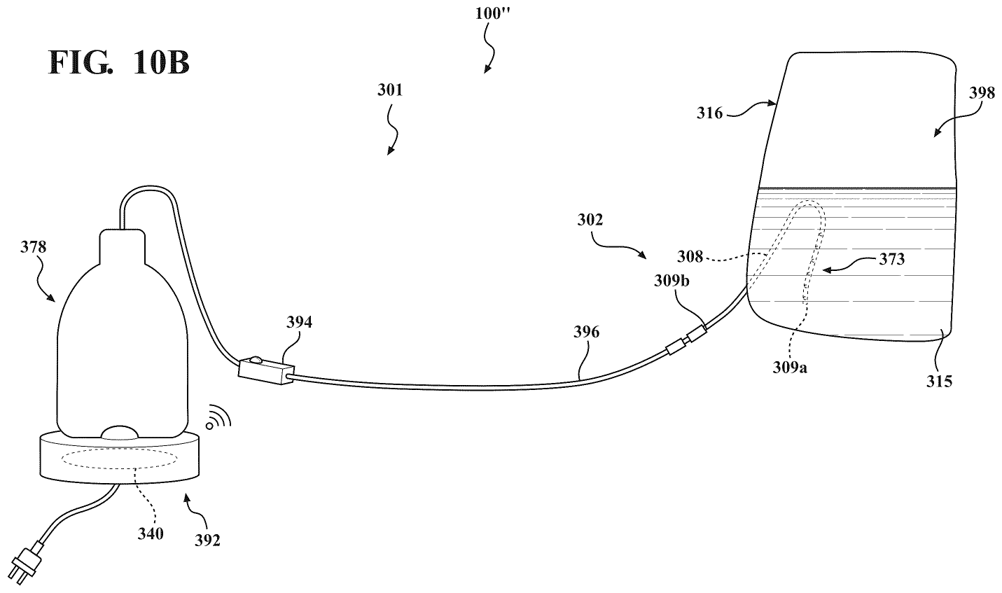

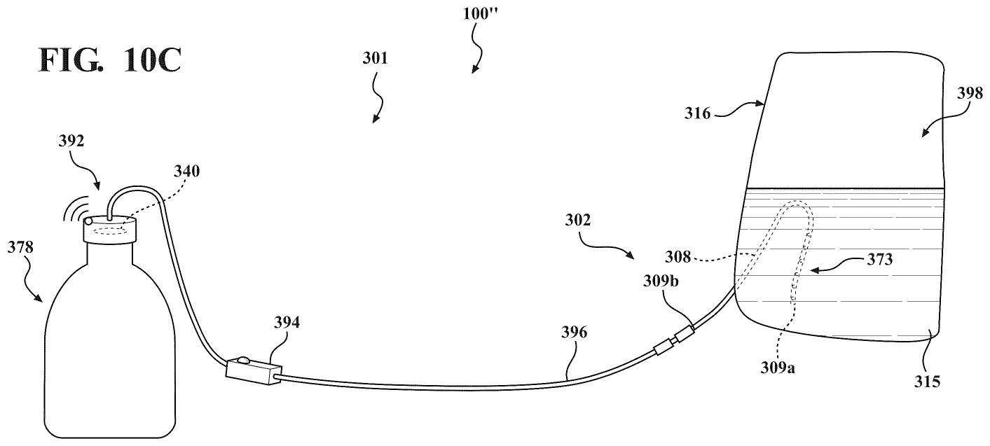

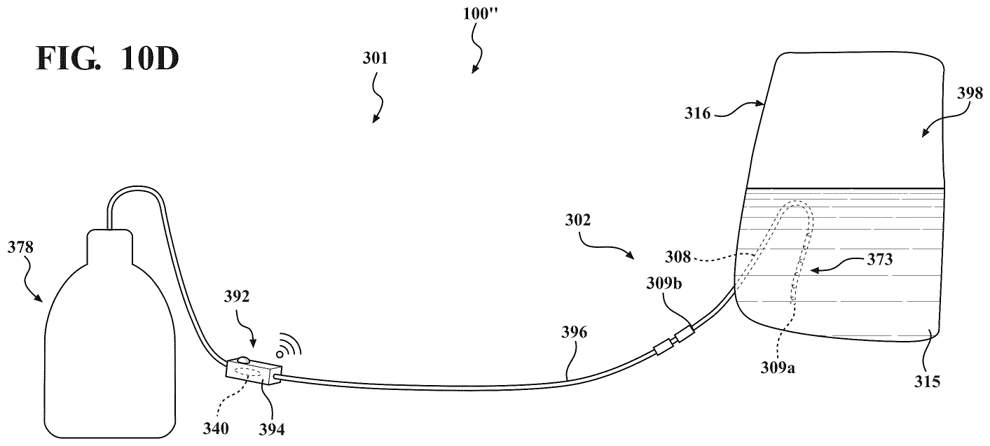

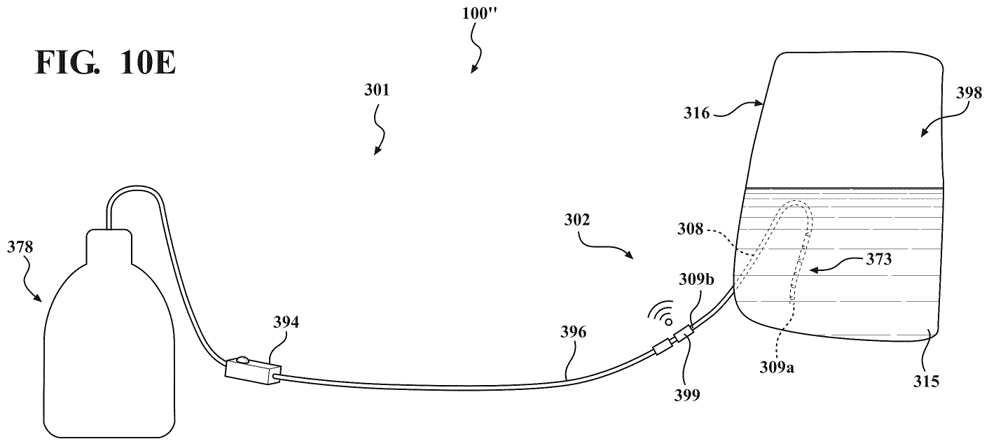

Referring now to FIGS. 10A-10E, a third aspect of the device monitoring system 100″ is generally depicted. In this aspect, the device monitoring system 100″ is a wireless drained volume and catheter device monitoring system 301, as discussed in greater detail herein. It is understood that the wireless drained volume and catheter device monitoring system 301 is similar to the catheter device monitoring system 201 with the exceptions of the features described herein. As such, like features will use the same reference numerals with a prefix “3” for the reference numbers. As such, for brevity reasons, these features will not be described again.

As illustrated, the wireless drained volume and catheter device monitoring system 301 may include the catheter device 302 that includes the catheter 308, which is fluidly coupled to the external fluid retention device 378. The wireless drained volume and catheter device monitoring system 301 may further include a dock 392 that includes a pressure sensor 340 and a flow control 394. The external fluid retention device 378 is a negative pressure retainer, such as a bottle. The volume of the fluids 315 (e.g., ascites, effusion, and the like) drained into the external fluid retention device 378 may be automatically measured by placing the dock 392 either in line with the fluid flow or out of flow, as discussed in greater detail herein. The volume of fluid drained may be determined through analysis of the rate of change of the pressure in the external fluid retention device 378.

That is, according to Boyle's Law, which states that at constant temperature, the volume of a given mass of a dry gas is inversely proportional to its pressure. In other words, P₁V₁=P₂V₂ where P₁=first pressure, P₂=second pressure, V₁=first volume, and V₂=second volume.

As illustrated in FIGS. 10A and 10D, the dock 392 is in-line with catheter 308 and is configured to measure a pressure within the catheter 308 without passing through the dock 392. For example, in FIG. 10A, the dock 392 includes the pressure sensor 340 configured to measure the pressure in the tubing connecting internal plastic coating tubing 396, fluidly coupled to the proximal end 309b of the catheter to the external fluid retention device 378. As such, the dock 392 may be easily reused. The dock 392 may wirelessly communicate with the controller 312, such as described above, using wireless protocols such as Bluetooth, cellular, or similar technology.

As such, the pressure sensor 340 of the dock 392 may be configured to detect changes in negative pressure of a pleural space 398 of the body 316 during inspiration and expiration. When the external fluid retention device 378 is sealed (e.g., closed by wither a roller clamp, seal on the bottle opening, and the like), and there is not changes in the negative pressure of the pleural space 398 detected by the pressure sensor 340, the controller 312 may determine that the internal plastic coating tubing 396 is not in contact with the pleural space 398 and there is either an occlusion within the catheter 308 and/or the catheter 308 has migrated.

Once the external fluid retention device 378 is unsealed, or open, pleural fluid (e.g., ascites fluid 315) begins entering the external fluid retention device 378 and the negative pressure begins to lower as the fluid fills the external fluid retention device 378. The pressure sensor 340 may sense the pressure in the external fluid retention device 378 and the volume drained may be determined by measuring the change in negative pressure over time and using Boyle's law to convert to a volume drained.

Now referring to FIG. 10B, in another embodiment, the external fluid retention device 378 may be positioned to sit on ort otherwise be in contact with the dock 392, which allows the pressure sensor 340 to integrate directly into the external fluid retention device 378. As such, the pressure sensor 340 of the dock 392 may automatically sense the volume drained based on changes in the external fluid retention device 378, in which the data is then transmitted (e.g., wirelessly) to the controller 312 for calculations, alerts, and display outputs.

Now referring to FIG. 10C, in a yet another embodiment, the dock 392 and pressure sensor 340 integrated therein may be coupled to and serve as a top of the external fluid retention device 378 at the end of drainage via the internal plastic coating tubing 396. As such, the pressure sensor may be configured to detect pressure changes or lack thereof such that the controller, based on the sensed data, determines and/or calculates occlusions and volume drained. That is, because the dock 392 is positioned above a foil seal on the external fluid retention device 378 in the vertical direction, occlusions may be detected before the foil seal is punctured and volume drained may be measured after the foil seal is punctured and fluid begins to drain.

Now referring to FIG. 10D, in yet another embodiment, the dock 392 and the pressure sensor 340 thereof may be integrated into the flow control 394. As such, when the dock 392 and the pressure sensor 340 is integrated into the flow control 394, the flow control 394 may be either disposable with the external fluid retention device 378 or may be reusable.

Now referring to FIG. 10E, in yet another embodiment, the dock 392 and the pressure sensor 340 thereof may be integrated into a catheter hub 399. Such an arrangement permits monitoring of pleural pressure even when the external fluid retention device 378 is not attached or fluidly coupled to the example wireless drained volume and catheter device monitoring system 301. Further, the amount of volume drained may also then be measured once the external fluid retention device 378 is fluidly coupled and flow is initiated.

Embodiments of the Present Disclosure May be Further Described with Respect to the Following Numbered Clauses:

1. A wireless monitoring system comprising: a controller configured to be positioned external to a subject; and a shunt device configured to be implanted within the subject, the shunt device having a pump chamber and a pair of limbs, the pump chamber or the pair of limbs comprising: one or more wireless sensors communicatively coupled to the controller, at least one or more wireless sensors configured to output a signal indicative of a total volume, a glucose amount, or an oxygenation within a fluid flowing in contact with the pump chamber or at least one of the pair of limbs, wherein the one or more wireless sensors wirelessly transmit data to the controller.

2. The wireless monitoring system of any preceding clause, wherein the one or more wireless sensors include at least one sensor configured to output a signal indicative of an acidity or basicity of the fluid in contact with the pump chamber or in contact with at least one of the pair of limbs.

3. The wireless monitoring system of any preceding clause, wherein the one or more wireless sensors include at least one sensor configured to output a signal indicative of a temperature of the fluid in contact with the pump chamber or in contact with at least one of the pair of limbs.

4. The wireless monitoring system of any preceding clause, wherein the shunt device is a peritoneovenous shunt configured to fluidly couple a peritoneal cavity to a venous system of the subject.

5. The wireless monitoring system of any preceding clause, wherein the shunt device is a pleuro-peritoneal shunt configured to fluidly couple a pleural cavity to a peritoneal cavity of the subject.

6. The wireless monitoring system of any preceding clause, wherein the one or more wireless sensors further include a pressure sensor positioned within the pump chamber or within at least one of the pair of limbs.

7. The wireless monitoring system of any preceding clause, wherein the one or more wireless sensors further include a pair of flow sensors, one of the pair of flow sensors is within one of the pair of limbs and the other one of the pair of flow sensors is within the other one of the pair of limbs to output a signal indicative of a differential fluid flow data.

8. The wireless monitoring system of any preceding clause, wherein the one or more wireless sensors are charged using inductive charging positioned external to the subject.

9. The wireless monitoring system of any preceding clause, a display device is communicatively coupled to the controller, wherein the controller displays the data on the display device.

10. The wireless monitoring system of any preceding clause, wherein the controller compares the data to a predetermined range and generates an alert when the controller determines the data is outside of predetermined range.

11. A pleuro-peritoneal shunt assembly for fluidly coupling a pleural cavity to a peritoneal cavity of a subject, the pleuro-peritoneal shunt assembly comprising: a controller configured to be positioned external to the subject, a shunt device configured to be embedded within the subject and in wireless communication with the controller, the shunt device comprising: a pair of limbs, and a pump chamber, one of the pair of limbs fluidly coupled to one side of the pump chamber and the other one of the pair of limbs fluidly coupled to an opposite side of the pump chamber, the pump chamber or the pair of limbs comprising: one or more wireless sensors communicatively coupled to the controller, at least one of the one or more wireless sensors configured to output a signal indicative of a total volume, a glucose amount, or an oxygenation of a fluid in contact with the pump chamber or within one of the pair of limbs, wherein the one or more wireless sensors wirelessly transmit a plurality of data to the controller.

12. The pleuro-peritoneal shunt assembly of any preceding clause, wherein the one or more wireless sensors communicatively coupled to the controller further include at least one sensor configured to output a signal indicative of an acidity or basicity of the fluid in contact with the pump chamber or in contact with at least one of the pair of limbs.

13. The pleuro-peritoneal shunt assembly of any preceding clause, wherein the one or more wireless sensors communicatively coupled to the controller further include a pressure sensor positioned within the pump chamber or within at least one of the pair of limbs.

14. The pleuro-peritoneal shunt assembly of any preceding clause, wherein the one or more wireless sensors communicatively coupled to the controller further include a pair of flow sensors, one of the pair of flow sensors is positioned within one of the pair of limbs and the other one of the pair of flow sensors is positioned within the other one of the pair of limbs to output a signal indicative of a differential fluid flow data.

15. The pleuro-peritoneal shunt assembly of any preceding clause, wherein the one or more wireless sensors communicatively coupled to the controller further include at least one sensor configured to output a signal indicative of a temperature of the fluid in contact with the pump chamber or in contact with at least one of the pair of limbs.

16. The pleuro-peritoneal shunt assembly of any preceding clause, a display device communicatively coupled to the controller, wherein the controller displays the data on the display device.

17. A peritoneovenous shunt assembly for fluidly coupling a peritoneal cavity to a venous system of a subject, the peritoneovenous shunt assembly comprising: a controller configured to be positioned external to the subject, a peritoneovenous shunt device configured to be embedded within the subject and in wireless communication with the controller, the peritoneovenous shunt device comprising: a pair of limbs, and a pump chamber, one of the pair of limbs fluidly coupled to one side of the pump chamber and the other one of the pair of limbs fluidly coupled to an opposite side of the pump chamber, the pump chamber or the pair of limbs comprising: one or more wireless sensors communicatively coupled to the controller, at least one of the one or more wireless sensors communicatively coupled to the controller configured to output a signal indicative of a total volume, a glucose amount, or an oxygenation of a fluid within the pump chamber or within at least one of the pair of limbs, wherein the one or more wireless sensors wirelessly transmit a plurality of data to the controller.

18. The peritoneovenous shunt assembly of any preceding clause, wherein the one or more wireless sensors communicatively coupled to the controller further comprise: at least one sensor configured to output a signal indicative of an acidity or basicity of the fluid within the pump chamber or within at least one of the pair of limbs; a pressure sensor positioned within the pump chamber or within the at least one of the pair of limbs; and at least one sensor configured to output a signal indicative of a temperature of the fluid within the pump chamber or within at least one of the pair of limbs.

19. The peritoneovenous shunt assembly of any preceding clause, wherein the one or more wireless sensors communicatively coupled to the controller further include a pair of flow sensors, one of the pair of flow sensors is positioned within one of the pair of limbs and the other one of the pair of flow sensors is positioned within the other one of the pair of limbs to output a signal indicative of a differential fluid flow data.

20. The peritoneovenous shunt assembly of any preceding clause, a display device communicatively coupled to the controller, wherein the controller displays the data on the display device.

21. A monitoring system comprising: a controller configured to be positioned external to a subject; and a catheter configured to be implanted within the subject, the catheter having a distal end and an opposite proximal end, an external valve portion positioned at the proximal end, the external valve portion comprising: one or more sensors communicatively coupled to the controller, at least one or more sensors configured to output a signal indicative of a total volume, a glucose amount, or an oxygenation within a fluid flowing in contact with the external valve portion or within the catheter, wherein the one or more sensors transmit data to the controller.

22. The monitoring system of any preceding clause, wherein the one or more sensors include at least one sensor configured to output a signal indicative of an acidity or basicity of the fluid in contact with the external valve portion or within the catheter.

23. The monitoring system of any preceding clause, wherein the one or more sensors include at least one sensor configured to output a signal indicative of a temperature of the fluid in contact with the external valve portion or within the catheter.

24. The monitoring system of any preceding clause, wherein the catheter is an indwelling peritoneal catheter configured to fluidly couple a peritoneal cavity of the subject to an external fluid retention device.

25. The monitoring system of any preceding clause, wherein the catheter is an indwelling pleural catheter configured to fluidly couple a pleural cavity of the subject to an external fluid retention device.

26. The monitoring system of any preceding clause, wherein the one or more sensors further include a pressure sensor positioned within the external valve portion or within the catheter.

27. The monitoring system of any preceding clause, wherein the catheter defines a pair of lumens extending between the distal end and the proximal end.

28. The monitoring system of any preceding clause, wherein one of the pair of lumens receives the fluid and the other one of the pair of lumens receives a plurality of electrical components associated with the one or more sensors.

29. The monitoring system of any preceding clause, wherein a display device is communicatively coupled to the controller, wherein the controller displays the data on the display device.

30. The monitoring system of any preceding clause, wherein the controller compares the data to a predetermined range and generates an alert when the controller determines the data is outside of predetermined range.

31. A pleural catheter assembly for fluidly coupling a pleural cavity to an external fluid retention device, the pleural catheter assembly comprising: a controller configured to be positioned external to a subject; and an indwelling pleural catheter comprising: a distal end, an opposite proximal end, and an external valve portion fluidly coupled to the proximal end, the external valve portion comprising: one or more sensors communicatively coupled to the controller, at least one of the one or more sensors configured to output a signal indicative of a total volume, a glucose amount, or an oxygenation of a fluid passing through the catheter, wherein the distal end is configured to be embedded within the subject, the external valve portion is configured to be in communication with the controller such that the one or more sensors transmit a plurality of data to the controller.

32. The pleural catheter assembly of any preceding clause, wherein the one or more sensors include at least one sensor configured to output a signal indicative of an acidity or basicity of the fluid passing through the catheter.

33. The pleural catheter assembly of any preceding clause, wherein the one or more sensors further include a pressure sensor positioned within the external valve portion or within the catheter positioned between the distal end and the proximal end.

34. The pleural catheter assembly of any preceding clause, wherein the catheter defines a pair of lumens extending between the distal end and the proximal end.

35. The pleural catheter assembly of any preceding clause, wherein the one or more sensors include at least one sensor configured to output a signal indicative of a temperature of the fluid within the catheter.

36. The pleural catheter assembly of any preceding clause, wherein a display device is communicatively coupled to the controller, wherein the controller displays the data on the display device.

37. A peritoneal catheter assembly for fluidly coupling a peritoneal cavity to an external fluid retention device, the peritoneal catheter assembly comprising: a controller configured to be positioned external to a subject, an indwelling peritoneal catheter comprising: a distal end, an opposite proximal end, and an external valve portion fluidly coupled to the proximal end, the external valve portion comprising: one or more sensors communicatively coupled to the controller, at least one of the one or more sensors configured to output a signal indicative of a total volume, a glucose amount, or an oxygenation of a fluid passing through the catheter, wherein the distal end is configured to be embedded within the subject, the external valve portion is configured to be in communication with the controller such that the one or more sensors transmit a plurality of data to the controller.

38. The peritoneal catheter assembly of any preceding clause, wherein the one or more sensors further comprise: at least one sensor of the one or more sensors is configured to output a signal indicative of an acidity or basicity of the fluid within the external valve portion or within the catheter; a pressure sensor of the one or more sensors is positioned within the external valve portion or within the catheter; and at least one sensor of the one or more sensors is configured to output a signal indicative of a temperature of the fluid within the catheter.

39. The peritoneal catheter assembly of any preceding clause, wherein the catheter includes a pair of lumens extending between the distal end and the proximal end.

40. The peritoneal catheter assembly of any preceding clause, wherein a display device is communicatively coupled to the controller, wherein the controller displays the data on the display device.

41. A monitoring system comprising: a controller configured to be positioned external to a subject; and a catheter having a distal end and an opposite proximal end, the distal end configured to be implanted within the subject, an external fluid retention device fluidly coupled to the proximal end of the catheter, the external fluid retention device having a negative pressure, and a dock device having a pressure sensor configured to sense a pressure within the catheter or within the external fluid retention device, the dock device in wireless communication with the controller to transmit data to the controller.

42. The monitoring system of any preceding clause, wherein the dock device is positioned within a fluid flow path between the catheter and the external fluid retention device.

43. The monitoring system of any preceding clause, wherein the dock device is positioned out of line of a fluid flow path between the catheter and the external fluid retention device.

44. The monitoring system of any preceding clause, further comprising: a tubing fluidly coupled to the proximal end of the catheter via a catheter hub and to the external fluid retention device; and a flow control coupled to the internal plastic coating tubing and configured to restrict a flow of fluid from the catheter to the internal plastic coating tubing.

45. The monitoring system of any preceding clause, wherein the dock device is positioned in line with a fluid flow path through the internal plastic coating tubing to sense the pressure of the internal plastic coating tubing.

46. The monitoring system of any preceding clause, wherein the controller is configured to, based on the sensed pressure by the dock device, determine whether there is an occlusion in the internal plastic coating tubing or in the catheter.

47. The monitoring system of any preceding clause, wherein the dock device is positioned as a top to the external fluid retention device.

48. The monitoring system of any preceding clause, wherein the dock device is positioned as a base to the external fluid retention device such that the external fluid retention device is position to rest on the dock device.

49. The monitoring system of any preceding clause, wherein the dock device is positioned within the flow control to sense the pressure within the internal plastic coating tubing.

50. The monitoring system of any preceding clause, wherein the dock device is positioned within the catheter hub to sense the pressure within the internal plastic coating tubing and the catheter.

It should now be understood that embodiments described herein are directed to a shunt device and a catheter device for draining ascites fluid and a monitoring device configured to detect the amount of pressure of the draining ascites fluid. The shunt device includes a pump chamber that includes at least one one-way valve, a first limb, and a second limb. The shunt device may be used in order to drain ascites fluid from the peritoneal cavity to the venous system or from the pleural cavity to the peritoneum, in order to remove the ascites fluid from the subject's body. A plurality of sensors may be positioned on or within the pump body, the first limb, the second limb, or a combination thereof, to transmit data (e.g., wirelessly) from within the body of the subject to a controller positioned outside of the body. Each of the plurality of sensors may be independently operated and may not rely on additional power, microcontrollers, or logic modules from within the shunt device. That is, each one of the plurality of sensors may include their own power source and ability to transmit data (e.g., wirelessly) to the controller positioned external to the body. The controller receives, interprets and displays the data. When certain data is above or below a predetermined threshold, the controller may alert a user, such as a physician. As such, the physician does not to actively monitor the various data for every subject.

The catheter device has a distal end, an opposite proximal end, and an external valve portion positioned at the proximal end. The external valve portion includes one or more sensors. The catheter device may be used in order to drain ascites fluid from the peritoneal cavity to the external fluid retention device or from the pleural cavity to the external fluid retention device, in order to remove the ascites fluid from the subject's body. A plurality of sensors may be positioned on or within the catheter, within the external valve portion, or a combination thereof, to transmit data (e.g., wired or wirelessly) related to the fluid gathered from within the body of the subject to a controller positioned outside of the body. Each of the plurality of sensors may be independently operated. The controller receives, interprets and displays the data. When certain data is above or below a predetermined threshold, the controller may alert a user, such as a physician. As such, the physician does not to actively monitor the various data for every subject.

The monitoring device is configured to detect the amount of pressure of the draining ascites fluid through the catheter, or other tubing, to a negative pressured external fluid retention device. The monitoring device, or dock device, includes an integrally formed pressure sensor configured to sense a pressure within the catheter or within the external fluid retention device. The dock device in wireless communication with the controller to transmit data to the controller. The controller receives, interprets and displays the data. When certain data is above or below a predetermined threshold, the controller may alert a user, such as a physician. As such, the physician does not to actively monitor the various data for every subject.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A wireless monitoring system comprising:
a controller configured to be positioned external to a subject; and
a shunt device configured to be implanted within the subject, the shunt device having a pump chamber and a pair of limbs, the pump chamber or the pair of limbs comprising:
one or more wireless sensors communicatively coupled to the controller, at least one or more wireless sensors configured to output a signal indicative of a total volume, a glucose amount, or an oxygenation within a fluid flowing in contact with the pump chamber or at least one of the pair of limbs wherein the one or more wireless sensors wirelessly transmit data to the controller.

2. The wireless monitoring system of claim 1, wherein the one or more wireless sensors include at least one sensor configured to output a signal indicative of an acidity or basicity of the fluid in contact with the pump chamber or in contact with at least one of the pair of limbs.

3. The wireless monitoring system of claim 1, wherein the one or more wireless sensors include at least one sensor configured to output a signal indicative of a temperature of the fluid in contact with the pump chamber or in contact with at least one of the pair of limbs.

4. The wireless monitoring system of claim 1, wherein the shunt device is a peritoneovenous shunt configured to fluidly couple a peritoneal cavity to a venous system of the subject.

5. The wireless monitoring system of claim 1, wherein the shunt device is a pleuro-peritoneal shunt configured to fluidly couple a pleural cavity to a peritoneal cavity of the subject.

6. The wireless monitoring system of claim 1, wherein the one or more wireless sensors further include a pressure sensor positioned within the pump chamber or within at least one of the pair of limbs.

7. The wireless monitoring system of claim 1, wherein the one or more wireless sensors further include a pair of flow sensors, one of the pair of flow sensors is within one of the pair of limbs and the other one of the pair of flow sensors is within the other one of the pair of limbs to output a signal indicative of a differential fluid flow data.

8. The wireless monitoring system of claim 1, wherein the one or more wireless sensors are charged using inductive charging positioned external to the subject.

9. The wireless monitoring system of claim 1, further comprising a display device communicatively coupled to the controller, wherein the controller displays the data on the display device.

10. The wireless monitoring system of claim 1, wherein the controller compares the data to a predetermined range and generates an alert when the controller determines the data is outside of predetermined range.

11. A pleuro-peritoneal shunt assembly for fluidly coupling a pleural cavity to a peritoneal cavity of a subject, the pleuro-peritoneal shunt assembly comprising:
a controller configured to be positioned external to the subject,
a pleuro-peritoneal shunt device configured to be embedded within the subject and in wireless communication with the controller, the pleuro-peritoneal shunt device comprising:
a pair of limbs, and
a pump chamber, one of the pair of limbs fluidly coupled to one side of the pump chamber and the other one of the pair of limbs fluidly coupled to an opposite side of the pump chamber, the pump chamber or the pair of limbs comprising:
one or more wireless sensors communicatively coupled to the controller, at least one of the one or more wireless sensors configured to output a signal indicative of a total volume, a glucose amount, or an oxygenation of a fluid in contact with the pump chamber or in contact with one of the pair of limbs,
wherein the one or more wireless sensors wirelessly transmit a plurality of data to the controller.

12. The pleuro-peritoneal shunt assembly of claim 11, wherein the one or more wireless sensors communicatively coupled to the controller further include at least one sensor configured to output a signal indicative of an acidity or basicity of the fluid in contact with the pump chamber or in contact with at least one of the pair of limbs.

13. The pleuro-peritoneal shunt assembly of claim 11, wherein the one or more wireless sensors communicatively coupled to the controller further include a pressure sensor positioned within the pump chamber or within at least one of the pair of limbs.

14. The pleuro-peritoneal shunt assembly of claim 11, wherein the one or more wireless sensors communicatively coupled to the controller further include a pair of flow sensors, one of the pair of flow sensors is positioned within one of the pair of limbs and the other one of the pair of flow sensors is positioned within the other one of the pair of limbs to output a signal indicative of a differential fluid flow data.

15. The pleuro-peritoneal shunt assembly of claim 11, wherein the one or more wireless sensors include at least one sensor configured to output a signal indicative of a temperature of the fluid in contact with the pump chamber or in contact with at least one of the pair of limbs.

16. The pleuro-peritoneal shunt assembly of claim 11, wherein a display device is communicatively coupled to the controller, wherein the controller displays the data on the display device.

17. A peritoneovenous shunt assembly for fluidly coupling a peritoneal cavity to a venous system of a subject, the peritoneovenous shunt assembly comprising:
a controller configured to be positioned external to the subject,
a peritoneovenous shunt device configured to be embedded within the subject and in wireless communication with the controller, the peritoneovenous shunt device comprising:
a pair of limbs, and
a pump chamber, one of the pair of limbs fluidly coupled to one side of the pump chamber and the other one of the pair of limbs fluidly coupled to an opposite side of the pump chamber, the pump chamber or the pair of limbs comprising:
one or more wireless sensors communicatively coupled to the controller, at least one of the one or more wireless sensors configured to output a signal indicative of a total volume, a glucose amount, or an oxygenation of a fluid within the pump chamber or within at least one of the pair of limbs,
wherein the one or more wireless sensors wirelessly transmit a plurality of data to the controller.

18. The peritoneovenous shunt assembly of claim 17, wherein the one or more wireless sensors communicatively coupled to the controller further comprise:
at least one sensor configured to output a signal indicative of an acidity or basicity of the fluid within the pump chamber or within at least one of the pair of limbs;
a pressure sensor positioned within the pump chamber or within the at least one of the pair of limbs; and
at least one sensor configured to output a signal indicative of a temperature of the fluid within the pump chamber or within at least one of the pair of limbs.

19. The peritoneovenous shunt assembly of claim 17, wherein the one or more wireless sensors communicatively coupled to the controller further include a pair of flow sensors, one of the pair of flow sensors is positioned within one of the pair of limbs and the other one of the pair of flow sensors is positioned within the other one of the pair of limbs to output a signal indicative of a differential fluid flow data.

20. The peritoneovenous shunt assembly of claim 17, wherein a display device is communicatively coupled to the controller, wherein the controller displays the data on the display device.

21.-50. (canceled)
